SEQUENCE LISTING (19 sequences)

SEQ ID NO: 1 - DNA, 20 bp, Mus musculus
gggcaaccgaggcaccagtc

SEQ ID NO: 2 - DNA, 20 bp, Mus musculus
cgcctcttggccttgtcccg

SEQ ID NO: 3 - DNA, 20 bp, Mus musculus
gagctgtgttcccggtgccc

SEQ ID NO: 4 - DNA, 24 bp, Mus musculus
ccgtgcttcctctggtaaaagcca

SEQ ID NO: 5 - DNA, 24 bp, Mus musculus
actcccaagtctgtgaggttccga

SEQ ID NO: 6 - DNA, 23 bp, Mus musculus
tgtcaaaagaatcagcggccggg

SEQ ID NO: 7 - DNA, 20 bp, Mus musculus
ggacttcgagcaagagatgg

SEQ ID NO: 8 - DNA, 20 bp, Mus musculus
ccgccagacagcactgtgtt

SEQ ID NO: 9 - DNA, 20 bp, Homo sapiens
gctttggccaagcccaggga

SEQ ID NO: 10 - DNA, 20 bp, Homo sapiens
tgctgggctccacctacccc

SEQ ID NO: 11 - DNA, 20 bp, Homo sapiens
ctcctcgggagccacacgca

SEQ ID NO: 12 - DNA, 20 bp, Homo sapiens
taggggagctggctgggtgg

SEQ ID NO: 13 - DNA, 20 bp, Mus musculus
tgaggctgcagcttgcctgg

SEQ ID NO: 14 - DNA, 20 bp, Mus musculus
accaagctacccctggccct

SEQ ID NO: 15 - DNA, 25 bp, Mus musculus
acctgttactttgggagtggcaagc

SEQ ID NO: 16 - DNA, 24 bp, Mus musculus
gtcgtcccagttggtaacaatgcc

SEQ ID NO: 17 - DNA, 27 bp, Mus musculus
atcgccttctatcgccttcttgacgag

SEQ ID NO: 18 - DNA, 21 bp, Mus musculus
gctacaatcccctgcagtacc

SEQ ID NO: 19 - DNA, 18 bp, Mus musculus
tgccttcttgcccttgtg

(19) United States
(12) Patent Application Publication
(10) Pub. No.: US 2013/0052162 A1
(43) Pub. Date: Feb. 28, 2013

(54) BOOSTING IMMUNE DEFENSE BY UPREGULATING CCAAT/ENHANCER BINDING PROTEIN EPSILON

(71) Applicants: H. Phillip Koeffler, Los Angeles, CA (US); George Y. Liu, Los Angeles, CA (US); Nils Thoennissen, Korschenbroich (DE); Pierre Kyme, Los Angeles, CA (US); Adrian F. Gombart, Eugene, OR (US)

(72) Inventors: H. Phillip Koeffler, Los Angeles, CA (US); George Y. Liu, Los Angeles, CA (US); Nils Thoennissen, Korschenbroich (DE); Pierre Kyme, Los Angeles, CA (US); Adrian F. Gombart, Eugene, OR (US)

(73) Assignee: CEDARS-SINAI MEDICAL CENTER, Los Angeles, CA (US)

(21) Appl. No.: 13/642,466

(22) PCT Filed: Apr. 20, 2011
(86) PCT No.: PCT/US11/33286

§ 371 Date: Oct. 19, 2012

Related U.S. Application Data
(60) Provisional application No. 61/326,143, filed on Apr. 20, 2010.

(51) Int. Cl. A61K 31/455; A61P 37/04; A61P 31/00; A61P 33/00; A61P 31/10; A61P 31/04; A61P 31/12; A61P 31/20; A61P 31/16; A61P 31/22; A61P 31/14; A61P 29/00; A61P 9/10; A61P 1/00; A61P 25/00; A61P 19/02; A61P 11/06; A61P 17/00; A61K 38/21

(52) U.S. Cl. 424/85.5; 424/278.1

(57) ABSTRACT

The present invention demonstrates the important role of C/EBPε in innate immune response against pathogens. Specifically, the inventors showed that in the absence of functional C/EBPε, mice are severely impaired in their ability to clear S. aureus infection. Neutrophils are particularly affected, and susceptibility to S. aureus can be rectified by treatment with interferon-gamma (IFN-γ). Importantly, increased activity of C/EBPε, either by induced overexpression of C/EBPE or by application of nicotinamide or an analog, derivative or salt thereof, dramatically enhances immune killing of S. aureus and leads to amelioration of infection.

### CROSS-REFERENCE TO RELATED APPLICATIONS

This application claims priority from U.S. Provisional Patent Application No. 61/326,143, filed on Apr. 20, 2010, which is incorporated herein by reference in its entirety.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant Nos. A1074832, R01CA026038-30, U54CA143930-01 and A1065604-04 awarded by the National Institutes of Health.

### FIELD OF THE INVENTION

This invention generally relates to methods of boosting immune defense against pathogens by upregulating CCAAT/enhancer binding protein epsilon.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Staphylococcus aureus in community and healthcare settings commonly causes serious and potentially life-threatening infections^{1,2,3}. Widespread use of antibiotics is responsible for the emergence and rapid spread of resistant pathogens, including methicillin-resistant S. aureus (MRSA)³, and highlights a pressing need for development of novel antimicrobial therapies.

Increasingly, novel therapeutics are identified by studying host and bacterial factors that play important roles in the immunopathology of infection. For example, the golden pigment of S. aureus is an important virulence factor that shields the pathogen from host oxidative killing, and the inventors have previously shown that blocking the biosynthesis of this pigment could be a strategy for treatment of S. aureus infection⁴¹. Conversely, among human genetic conditions that alter susceptibility to S. aureus infection is the neutrophil-specific granule deficiency (SGD), a rare hematologic disorder characterized by a significantly defective immunity^{4,7}. Patients with SGD present with functional defects in neutrophils, as well as monocytes/macrophages, and suffer from recurrent life-threatening bacterial infections, including S. aureus, Pseudomonas aeruginosa, and Klebsiella pneumoniae. Therefore, understanding of the host immune mechanisms conferring susceptibility to S. aureus could lead to identification of immune modulatory strategies⁴⁷.

SGD is caused by mutations of the gene encoding the transcription factor CCAAT/enhancer binding protein epsilon (C/EBPε)^{8,9}. C/EBPε, which was originally cloned by the inventors' group and others^{10,11}, is a nuclear transcription factor expressed specifically in myeloid cells. C/EBPε serves as an important regulator of the terminal differentiation and functional maturation of neutrophils and macrophages^{11,17}, both crucial components of the innate immune system. Neutrophils from C/EBPε-deficient (C/EBPε^{-/-}) mice display aberrant phagocytosis, respiratory burst, and bactericidal activities, similar to neutrophils from individuals with SGD^{8,9,11-15}. Importantly, in the presence of other C/EBP family members, C/EBPε^{-/-} neutrophils lack expression of all secondary (specific) granule proteins, including antimicrobials such as lactoferrin (LF), cathelicidin, neutrophil gelatinase, and collagenase. In addition, murine and human monocytes/macrophages with impaired expression of C/EBPε display signs of immaturity, impaired phagocytosis, and altered myelomonocytic-specific gene expression^{7,13,16,17}.

It has been demonstrated that the activity of the highly conserved family member, C/EBPβ is regulated in part by its acetylation and deacetylation¹⁸. Therefore, while not wishing to be bound by any one particular theory, it is possible that modification of acetylation could be important for the regulation of the transcription factor C/EBPε and its downstream antimicrobial targets. Histone-deacetylase (HDAC) inhibitors are essential epigenetic regulators of transcription that modify acetylation of histones and non-histone transcription factors¹⁹⁻²³. These inhibitors can block the activity of certain HDACs and induce histone acetylation, leading to the relaxation of chromatin structure, enhanced accessibility of transcriptional machinery to DNA, and increased gene transcription^{19,21}. HDAC inhibitors may also induce acetylation of non-histone proteins, resulting in changes in their activity and of downstream target genes^{22,23}. Nicotinamide (NAM), also referred to as vitamin B3, is the amide of nicotinic acid, and is well known to act as a competitive inhibitor of class III HDACs²⁴⁻²⁷. Complex immunomodulatory effects of NAM have been reported in mammalian cells⁴⁸.

There is a need in the art to develop new therapeutic strategies to boost immune defense.

### SUMMARY OF THE INVENTION

In one embodiment, the present invention provides a method, including: providing a composition that upregulates the expression of CCAAT/enhancer binding protein epsilon (C/EBPε), and administering a therapeutic dose of the composition to an individual having an infection caused by a pathogen, whereby an enhanced immune response to the infection results in the individual. In certain embodiments, the composition comprises vitamin B3 or an analog, derivative or salt thereof. In certain embodiments, the pathogen includes: parasites, fungi, bacteria, viruses, or combinations thereof. In certain embodiments, the pathogen is selected from the group consisting of: Staphylococcus aureus (S. aureus), methicillin-resistant S. aureus, Vancomycin resistant Eznterococcus, C. difficile, B. cepacia, influenza, rhino virus, Epstein barr virus, cytoinegalovirus, adenovirus, parainfluenza virus, rotavirus, candida, ESBL gramin negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof. In certain embodiments, the individual is a mammal. In certain embodiments, the individual is a human.

In another embodiment, the present invention provides a method, including: providing a composition that upregulates the expression of CCAAT/enhancer binding protein epsilon (C/EBPε), and administering a prophylactic dose of the composition to an individual, whereby the likelihood of developing a severe pathogenic infection in the individual is reduced. In certain embodiments, the composition comprises vitamin B3 or an analog, derivative or salt thereof. In certain embodiments, the pathogenic infection is caused by a pathogen selected from the group consisting of: parasites, fungi, bacteria, viruses, or combinations thereof. In certain embodiments, the pathogenic infection is caused by a pathogen selected from the group consisting of: Staphylococcus aureus (S. aureus), methicillin-resistant S. aureus, Vancomycin resistant Enterococcus, C. difficile, B. cepacia, influenza, rhinovirus, Epstein barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof. In certain embodiments, the individual is a mammal. In certain embodiments, the individual is a human. In certain embodiments, the composition is administered as part of a parenteral nutrition regimen. In certain embodiments, the individual is a neonate or other patient that cannot eat on his or her own.

In another embodiment, the present invention provides a method, including: providing interferon-gamma, and administering a therapeutic dose of interferon-gamma to an individual having a pathogenic infection and a defective innate immune response thereto, whereby the severity of the pathogenic infection is reduced. In certain embodiments, the pathogen is selected from the group consisting of: parasites, fungi, bacteria, viruses, or combinations thereof. In certain embodiments, the pathogen is selected from the group consisting of: Staphylococcus aureus (S. aureus), methicillin-resistant S. aureus, Vancomycin resistant Enterococcus, C. difficile, B. cepacia, influenza, rhinovirus, Epstein barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof. In certain embodiments, the individual is a mammal. In certain embodiments, the individual is a human. In certain embodiments, the individual has neutrophil-specific granule deficiency.

In another embodiment, the invention discloses a method, including: providing interferon-gamma, and administering a prophylactic dose of interferon-gamma to an individual with a defective innate immune response to a pathogen, whereby the likelihood of developing a severe pathogenic infection is reduced. In certain embodiments, the pathogen is selected from the group consisting of: parasites, fungi, bacteria, viruses, or combinations thereof. In certain embodiments, the pathogen is selected from the group consisting of: Staphylococcus aureus (S. aureus), methicillin-resistant S. aureus, Vancomycin resistant Enterococcus, C. difficile, B. cepacia, influenza, rhinovirus, Epstein barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof. In certain embodiments, the individual is a mammal. In certain embodiments, the individual is a human. In certain embodiments, the individual has neutrophil-specific granule deficiency.

In another embodiment, the invention provides a method, including: providing a composition that upregulates the expression of CCAAT/enhancer binding protein epsilon (C/EBPε), and administering a therapeutic dose of the composition to an individual having an inflammatory condition, whereby an increased anti-inflammatory response results in the individual. In certain embodiments, the composition is Vitamin B3 or an analog, derivative or salt thereof. In certain embodiments, the upregulation of C/EBPEε increases interleukin 10 (IL-10) function. In certain embodiments, the increased IL-10 function results in anti-inflammatory mediation of an inflammatory and/or autoimmune condition selected from the group consisting of: atherosclerosis, inflammatory bowel diseases, multiple sclerosis, rheumatoid arthritis, asthma, bacterial sepsis, Kawasaki's disease, atopic dermatitis, and other rheumatologic conditions. In certain embodiments, the invention teaches a kit including: a volume of a composition that upregulates the expression of CCAAT/enhancer binding protein epsilon (C/EBPε), and instructions for the use of said composition in the treatment of a disease condition in a mammal.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 demonstrates, in accordance with an embodiment of the invention, C/EBPε is important for clearance of S. aureus in a murine model of skin infection. (a-e) WT and C/EBPε^{-/-} mice (n=8 mice/group) were injected s.c. with two different inocula of S. aureus (left flank, ˜2×10⁷ CFU; right flank, ˜1×10⁸ CFU). (a) Overall body weight (mean±s.e.m) during 6 days p.i., presented as percent of original weight. P<0.001 (two-way ANOVA). (b) Area of skin lesions. P<0.001 (two-way ANOVA). Shown on right are representative images of skin lesions on day 6. (Arrows point to lesions from ˜2×10⁷ inoculum (left) and ˜1×10⁸ inoculum (right) (c) CFU recovered from skin lesions, spleen, and kidneys on day 6 p.i. Dashed line indicates the limit of detection. FIG. 8 shows the area of skin lesions and CFU from mice infected s.c. with ˜1×10⁸ CFU S. aureus. (d) Neutrophil and macrophage counts from H&E staining of two representative skin lesions (per genotype) after 24 h of infection. Microscopy images show skin lesions from WT and C/EBPε^{-/-} mice 24 h after infection. Arrows indicate the area of infection. (e) CXCL1 and CXCL2 measured from the skin lesions (n=10 lesions/group) at 24 h p.i. (f) Survival of 1.2×10⁴ CFU/mL S. aureus in peripheral blood drawn from WT and C/EBPε^{-/-} mice (n=5/group). Blood was pooled separately and inoculated in triplicate for 1 h. FIG. 9 shows data for whole blood survival assay performed with ˜5×10³ CFU/mL S. aureus. Data in d, e, and f, are means±s.d. The bar in b and c indicates mean. *** P<0.001.

FIG. 2 demonstrates, in accordance with an embodiment of the invention, depletion of neutrophils improves the outcome of bacterial infection in C/EBPε^{-/-} mice. (a-b) WT and C/EBPε^{-/-} mice (n==6/group) were treated with anti-PMN antibodies or normal serum daily for 4 days, and infected s.c. on day 2 with ˜4×10⁶ CFU S. aureus. (a) Area of skin lesions (mean±s.e.m; P<0.001 for all indicated comparisons; two-way ANOVA). (b) CFU recovered from the skin lesions, spleen, and kidneys on day 4. FIG. 12 presents the skin lesion area and the respective CFU following s.c. infection of mice with ˜2×10⁷ CFU S. aureus. The bar indicates mean. * P<0.05; ** P<0.01. (c) Bacterial clearance by peripheral whole blood and cell-free plasma from WT and C/EBPε^{-/-} mice. Whole blood or plasma from WT mice (n=8) and C/EBPε^{-/-} mice (n=7) was inoculated with 4.1×10³ CFU/mL S. aureus for 1.5 h. Similar data was observed using a higher bacterial inoculum of 7.5×10³ CFU/mL S. aureus (not shown). Data are means±s.d.; * P<0.05; *** P<0.001. Representative histology of infected skin lesions from WT and C/EBPε^{-/-} mice showing neutrophils with intracellular S. aureus (arrows).

FIG. 3 demonstrates, in accordance with an embodiment of the invention, IFN-γ administration enhances clearance of S. aureus in C/EBPε^{-/-} mice. (a-c) C/EBPε^{-/-} mice were injected 5,000 U (i.p.) of recombinant murine IFN-γ daily for 5 days, and infected s.c. with S. aureus (left flank: ˜2×10⁷ CFU: right flank: ˜4×10⁶ CFU) on day 2. Infected WT and C/EBPε^{-/-} control mice were each treated with PBS. (a) CFU recovered from the skin lesions after infection with ˜4×10⁶ CFU S. aureus. Dashed line indicates the limit of detection. ** P<0.01. (FIG. 13a shows CFU recovered from the skin lesions of mice infected s.c. with ˜2×10⁷ CFU S. aureus). (b) Percentage weight loss (n=5 mice/group). Data are means±s.e.m (P<0.001 for all indicated comparisons; two-way ANOVA). (c) Lesion size in C/EBPε^{-/-} mice. P>0.05 between PBS- and IFN-γ-treated mice (two-way ANOVA). Also shown are images of representative lesions, indicated by arrows. The bar in a and c indicates mean. (FIG. 12b shows the skin lesion areas caused by s.c. infection with ˜2×10⁷ CFU S. aureus).

FIG. 4 demonstrates, in accordance with an embodiment of the invention, induced overexpression of C/EBPε is associated with increased killing of S. aureus by macrophages. The pro-monocytic cell line U937 was stably transfected with either the zinc-inducible C/EBPε-expression vector (pMTε) or vector control (pMT), differentiated to macrophages using PMA, and treated with PBS or zinc. (a) The PMA-derived macrophages were then infected with a methicillin-sensitive strain of S. aureus (Pig1) at multiple different MOIs (bacteria:macrophage) for 24 h. ** P<0.01; *** P<0.001. Data (means±s.d.) are representative of three independent experiments performed in triplicate. No significant difference was observed between the four control groups of infected macrophages (non-transfected; pMT with/without zinc; pMTε without zinc; P>0.05; one-way ANOVA). These findings were repeated in PMA-derived macrophages infected with MRSA (FIG. 14). Addition of zinc alone had no effect on the viability and growth of S. aureus cultures (FIG. 15). (b) Western blot revealed a 6.5-fold increase in C/EBPε protein expression in lysates from the infected zinc-treated macrophages carrying pMTε, compared to the controls.

FIG. 5 demonstrates, in accordance with an embodiment of the invention, the activity of C/EBPε in myeloid cells can be increased in vitro and in vivo using the HDAC inhibitor NAM. (a) Effect of NAM on histone acetylation in BMDM. WT BMDM were treated with 1 mM NAM for 6 h and 12 h followed by chromatin immunoprecipiation (ChIP) using an antibody against acetylated histone H3 (Ac-H3) or IgG (negative control). The samples were analyzed by PCR using primers specific for the CEBPE promoter region. The input chromatin was included as a positive control using primers for the βl-actin gene. After 12 h of treatment with NAM, histone acetylation increased 5-fold compared to untreated BMDM. (b) mRNA expression of CEBPE and the downstream target genes CAMP and LF after treatment of BMDM with 1 mM NAM. On the right, Western blot shows 3-fold increase in expression of C/EBPε after addition of 1 mM NAM to WT BMDM for 12 h compared to the control. (c) Effect of NAM on histone acetylation in human PMNs. Peripheral blood from three healthy human volunteers was treated with 1 mM NAM for 6 h or 12 h, and the PMNs subsequently extracted. ChIP assay (representative gel shown) revealed increased (5-fold) histone H3 acetylation specifically in the CEBPE promoter region after 12 h treatment. (FIG. 16 outlines additional ChIP assay results using WT BMMC treated with NAM ex vivo). (d) Effect of NAM on LF reporter gene activity. A reporter assay was performed with U937 pro-monocytic cells transiently transfected with either a reporter construct containing a cDNA fragment of the LF-promoter (LAC-LUC), or control vehicle (pGL3). U937 cells were treated with 1 mM NAM or PBS for 16 h. (e) Effect of NAM on acetylation of C/EBPε protein in BMDM. Lysates from BMDM treated with 1 mM NAM for 6 h were subjected to immunoprecipitation (IP) with an antibody against pan-acetylated lysine residues (acetyl-Lys), followed by Western blot (WB) with an antibody against C/EBPε. Acetylation of C/EBPε increased 4-fold in the NAM-treated samples. (FIG. 17 contains additional IP data on BMDM treated with 10 mM NAM, and BMMC treated with both 1 mM and 10 mM NAM). (f) mRNA expression of CEBPE, CAMP, and LF in BMMC isolated from NAM-treated WT mice. Non-infected WT mice (n=4) received either NAM (250 mg/kg/day, i.p.) or PBS (control). After 72 h, BMMC were extracted and real-time RT-PCR expression analysis was performed. Data in b, d, and f are means±s.d. Fold-changes of illustrated gels (a and c) and blots (b and e) were measured by densitometry.

FIG. 6 demonstrates, in accordance with an embodiment of the invention, NAM improves the outcome of S. aureus infection in mice and in human blood and is dependent on C/EBPε, in accordance with an embodiment of the invention. (a) Effect of NAM on clearance of S. aureus by WT and C/EBPε^{-/-} murine peripheral blood. Blood from WT and C/EBPε^{-/-} mice (n===6/group) was pooled and treated with either NAM (1 mM) or PBS. After 24 h, triplicate blood samples were inoculated with ˜1×10⁴ CFU/mL S. aureus for 1 h and 3 h. Significantly less CFU were recovered from NAM- versus PBS-treated blood of WT mice after 1 h and 3 h of infection (* P<0.05; *** P<0.001; paired t-test). In contrast, CFU recovered from the NAM-treated blood of C/EBPε^{-/-} mice were not statistically different to CFU from PBS-treated blood at each time point (P>0.05; paired t-test). Images illustrate the resulting CFU obtained after 3 h from the blood of WT and C/EBPε^{-/-} mice treated with PBS or NAM. (FIG. 18 shows additional data from blood samples inoculated with 2.3×10³ CFU/mL or 5.2×10³ CFU/mL S. aureus). (b-d) Effect of NAM on in vivo clearance of S. aureus by WT and C/EBPε^{-/-} mice. WT (n=9) or C/EBPε^{-/-} mice (n=7) were treated daily with NAM (250 mg/kg/day, i.p.) or with PBS (control), beginning 24 h prior to systemic (i.p.) infection with ˜1×10⁷ CFU S. aureus. (b) CFU count in spleen and kidneys of WT mice at 48 h p.i. Dashed line indicates the limit of detection. The bar indicates mean. *** P<0.001. (c) mRNA levels of CEBPE, CAMP, and LF in BMMC at 48 h p.i. Representative data (means±s.d.) of 4 out of the 9 mice per treatment group are shown. (d) CFU count in spleen and kidneys of C/EBPε^{-/-} mice at 48 h p.i. Comparing PBS and NAM-treated C/EBPε^{-/-} mice, P>0.05 for both spleen and kidneys. One out of a total of seven mice from each of the two treatments groups died and was not included in data collection. The bar indicates mean. (e) Effect of NAM in WT mice already infected with S. aureus. Animals (n=9/group) were systemically infected i.p. with ˜2.0×10⁷ CFU S. aureus, and treated daily with either NAM (250 mg/kg/day, i.p.) or with PBS (control), beginning 12 h p.i.; CFU count in spleen and kidneys of WT mice at 60 h p.i. Dashed line indicates the limit of detection. The bar indicates mean. *** P<0.001.

FIG. 7 demonstrates, in accordance with an embodiment of the invention, NAM improves the outcome of S. aureus infection in human blood. (a-b) Effect of NAM on clearance of S. aureus by human peripheral blood. Whole blood obtained from 12 healthy human volunteers was pretreated with NAM (1 mM or 10 mM) or PBS for 24 h, and subsequently inoculated in triplicate with S. aureus for 1 h and 3 h. (a) Bacterial counts (means±s.d.) recovered from the blood of 4 volunteers after inoculation with 6.5×10³ CFU/mL S. aureus. Significantly less CFU were recovered from NAM- versus PBS-treated blood after 1 h and 3 h of infection. * P<0.05; ** P<0.01; all paired t-tests. FIG. 20 contains further data from the blood of the same 4 volunteers as well as from 5 additional volunteers, using different inocula of S. aureus. Under similar experimental settings, NAM- and PBS-treated blood from an additional 3 human volunteers was inoculated with S. aureus and yielded consistent results (data not shown). (b) Levels of C/EBPε in human PMNs isolated from NAM-treated blood. Non-infected blood from 3 healthy human volunteers (#1-3) was treated with 1 mM NAM for 12 or 24 h. As indicated by Western blot, levels of C/EBPε were 2- to 4-fold increased (by densitometry) in the PMN lysates recovered from the NAM-treated versus PBS-treated blood.

FIG. 8 demonstrates, in accordance with an embodiment of the invention, C/EBPε^{-/-} mice are highly susceptible to S. aureus subcutaneous challenge. (a) Graph shows the significantly larger area of skin lesions from C/EBPε^{-/-} compared to WT mice infected s.c. with ˜1×10⁸ CFU S. aureus (P<0.001; two-way ANOVA; the bar represents mean). (b) Higher CFU were recovered from these skin lesions of C/EBPε^{-/-} mice on day 6 p.i. The bar indicates mean. (P<0.001)

FIG. 9 demonstrates, in accordance with an embodiment of the invention, blood derived from C/EBPε^{-/-} mice is defective in clearance of S. aureus. Peripheral blood drawn from WT or C/EBPε^{-/-} mice (n=5 mice/group) was pooled and inoculated in triplicate with ˜5×10³ CFU/mL S. aureus for 1 h, at which time the surviving CFU were quantified and compared between the two groups (* P<0.05). Data are means±s.d.

FIG. 10 clearance of S. aureus from BMDM of C/EBPε^{-/-} mice is impaired but treatment with IFN-γ can compensate for this defect. BMDM (5×10⁴ cells) harvested from C/EBPε^{-/-} mice were incubated with recombinant murine IFN-γ (2001/mL) for 48 h. IFN-γ treated and control macrophages were then infected with S. aureus at a MOI of 2.5:1 (bacteria:macrophage) for 30 min at which time gentamicin was added to the culture media for 24 h. A greater number of intracellular bacteria was recovered from PBS-treated C/EBPε^{-/-} macrophages compared to WT controls (* P<0.05). IFN-γ treatment reduced the number of S. aureus CFU recovered from C/EBPε^{-/-} macrophages (* P<0.05). There was no difference in CFU recovered from WT control and IFN-γ-treated C/EBPε^{-/-} macrophages at 24 h (P>0.05). Data (means±s.d.) are representative of two independent experiments performed in triplicate.

FIG. 11 demonstrates, in accordance with an embodiment of the invention, mice heterozygous for C/EBPε are not more susceptible to S. aureus compared to WT mice. (a-d) WT mice and mice heterozygous for the epsilon gene (C/EBPε^{+/-}) (n=7 mice/group) were injected s.c. with two different inocula of S. aureus (left flank, 1×10⁸ CFU; right flank, 2×10⁷ CFU). (a) Left: Size of skin lesions (measured daily) from C/EBPε^{+/-} mice compared to WT mice infected s.c. with ˜2×10⁷ CFU S. aureus (P>0.05; two-way ANOVA). Right: No difference in CFU recovered from these skin lesions on day 6 p.i. (P>0.05). (b) Left: Size of skin lesions from C/EBPε^{+/-} mice compared to WT mice infected s.c. with ˜1×10⁸ CFU S. aureus (P>0.05; two-way ANOVA). Right: No difference in CFU recovered from these skin lesions on day 6 p.i. (P>0.05). (c) Images of the skin lesions (arrows) are representative of the phenotype between WT and C/EBPε^{+/-} mice 6 days after s.c. infection with S. aureus. (d) No differences in CFU were detected in the spleens or kidneys of WT and C/EBPε^{+/-} mice on day 6 p.i. (P>0.05; dashed line indicates the limit of detection). The bar indicates mean.

FIG. 12 demonstrates, in accordance with an embodiment of the invention, depletion of neutrophils leads to improved clearance of S. aureus and smaller skin lesion sizes in C/EBPε^{-/-} mice. Left: The area of skin lesions caused by an inoculum of ˜2×10⁷ CFU. Data are means±s.e.m (P<0.001 for all indicated comparisons; two-way ANOVA). Right: CFU recovered from the skin lesions on day 4 following s.c. infection of mice with 2×10⁷ CFU S. aureus. * P<0.05; ** P<0.01. The bars indicate means.

FIG. 13 demonstrates, in accordance with an embodiment of the invention, IFN-γ promotes S. aureus clearance in C/EBPε^{-/-} mice. (a) Higher CFU were recovered from the skin lesions (˜2×10⁷ CFU s.c. inoculum) of PBS-treated C/EBPε^{-/-} mice versus PBS-treated WT mice (* P<0.05). Treatment of C/EBPε^{-/-} mice with IFN-γ (compared to PBS) resulted in lower numbers of bacteria in the skin lesions (* P<0.05). Comparable CFU were recovered from the skin of IFN-γ-treated C/EBPε^{-/-} mice and PBS-treated WT mice (P>0.05). (b) The systemic application of IFN-γ to C/EBPε^{-/-} mice had no effect on the overall lesion area resulting from s.c. infection with ˜2×10⁷ CFU S. aureus (P>0.05 compared to control; two-way ANOVA). The bar indicates mean.

FIG. 14 demonstrates, in accordance with an embodiment of the invention, induced overexpression of C/EBPε promotes macrophage killing of S. aureus in vitro. The PMA-derived macrophages were infected with an MRSA S. aureus strain (LAC) at multiple different MOIs (bacteria:macrophage) for 24 h. ** P<0.01; *** P<0.001. Data (means±s.d.) are representative of three independent experiments performed in triplicate. No significant difference was observed between the four control groups of infected macrophages (non-transfected; pMT with/without zinc; pMTε without zinc; P>0.05; one-way ANOVA).

FIG. 15 demonstrates, in accordance with an embodiment of the invention, zinc does not have direct anti-staphylococcal activity. S. aureus strains (Pig1) (MSSA) or LAC (MRSA) (˜5×10⁵ CFU) were incubated with or without 100 μM Zn₂SO₄ (standard concentration used in the assays) in RPMI 1640 with 10% FBS, and CFU were enumerated after 24 h. Data are means±s.d. The assay was performed twice in triplicate.

FIG. 16 demonstrates, in accordance with an embodiment of the invention, NAM increases histone acetylation at the CEBPE promoter region. BMMC harvested from VT mice were treated with 1 mM NAM for 6 h and 12 h, followed by ChIP analysis using an antibody against acetylated histone H3 (Ac-H3) or IgG (negative control). The samples were analyzed by RT-PCR using primers specific for the CEBPE promoter region, and the input chromatin was included as a positive control using primers for the β-actin gene. By 12 h, histone acetylation increased 3-fold compared to untreated BMMC. Data are representative of two independent experiments performed in triplicate (n=3 mice). Fold-changes were measured by densitometry.

FIG. 17 demonstrates, in accordance with an embodiment of the invention, NAM increases acetylation of C/EBPε. (a) BMDM from WT mice were treated with 10 mM NAM for 6 h and subjected to immunoprecipitation (IP) with an antibody against pan-acetylated lysines (acetyl-Lys), followed by Western blot (WB) with an antibody against C/EBPε. Acetylation of C/EBPε increased 3-fold in the NAM-treated samples. (b) Blots display IP results from BMMC treated with 1 mM (Left) and 10 mM (Right) NAM. Acetylation of C/EBPε increased 3-fold in the 1 mM NAM treated samples, and 2-fold in the 10 mM treated samples. Data are representative of two independent experiments performed in triplicate (n===3 mice). Fold-changes were measured by densitometry.

FIG. 18 demonstrates, in accordance with an embodiment of the invention, NAM shows C/EBPε-dependent clearance of S. aureus from murine blood. Peripheral blood from WT and C/EBPε^{-/-} mice (n=6/group) was pooled and treated with either NAM (1 mM) or PBS. After 24 h, triplicate blood samples were inoculated with (a) 2.3×10³ CFU/mL or (b) 5.2×10³ CFU/mL S. aureus for 1 h and 3 h. Significantly less CFU were recovered from NAM- versus PBS-treated blood of WT mice after 1 h and 3 h of infection (*P<0.005. ** P<0.01; *** P<0.001; paired t-tests). In contrast, CFU recovered from the NAM-treated blood of C/EBPε^{-/-} mice were not statistically different to CFU from PBS-treated blood at each time point (P>0.05; paired t-test). Data are means±s.d.

FIG. 19 demonstrates, in accordance with an embodiment of the invention, preincubation of mouse blood with NAM for 4 h was not sufficient to promote clearance of S. aureus. Peripheral blood from WT mice (n==3 mice/group) was pooled, and treated with either NAM (1 mM) or PBS. After 4 h of pretreatment, triplicate blood samples were inoculated with S. aureus at 4×10³ CFU/mL (Left), 6.3×10³ CFU/mL (Middle), or 1.38×10⁴ CFU/mL (Right) for 1 h and 3 h, at which time surviving CFU were quantitated. Similar CFU were recovered from NAM-treated blood and PBS-treated blood at each time point (all P>0.05; paired t-tests). Data are means±s.d. The assay was repeated and yielded similar results (data not shown).

FIG. 20 demonstrates, in accordance with an embodiment of the invention, NAM enhances S. aureus clearance from the blood of human volunteers. (a) Bacterial counts (means±s.d.) recovered from the blood of 4 human volunteers after the inoculation with either 4×10³ CFU/mL (Left) or 1.3×10⁴ CFU/mL (Right) S. aureus. Significantly less CFU were recovered from NAM- versus PBS-treated blood after 1 h and 3 h of infection. * P<0.05; ** P<0.01; all paired t-tests. (b) Similar findings were observed using peripheral blood from 5 human volunteers inoculated with 2.5×10³ CFU/mL (Left) 5.3×10³ CFU/mL (Middle) and 1.3×10⁴ CFU/mL S. aureus (Right). * P<0.05; ** P<0.01; *** P<0.001; all paired t-tests.

FIG. 21 demonstrates, in accordance with an embodiment of the invention, NAM enhances clearance of K. pneumoniae and P. aeruginosa from the blood of human volunteers. (a) Bacterial counts (means±s.d.) recovered from the peripheral blood of 5 human volunteers after the inoculation with either 4.9×10³ CFU/mL (Left) or 1.2×10⁴ CFU/mL (Right) K. pneumoniae. Significantly less CFU were recovered from NAM- versus PBS-treated blood after 3 h of infection. ** P<0.01; *** P<0.001; all paired t-tests. (b) Similar findings were observed using peripheral blood from 5 human volunteers inoculated with 5.2×10³ CFU/mL (Left) or 1.2×10⁴ CFU/mL (Right) P. aeruginosa. * P<0.05; ** P<0.01; all paired t-tests. Data are means±s.d.

FIG. 22 demonstrates, in accordance with an embodiment of the invention, NAM does not have direct anti-staphylococcal activity. (a) NAM (1 mM and 10 mM, in either THB or PBS) or THB/PBS (control) was inoculated in triplicate with S. aureus (˜1×10⁴ CFU/mL in THB or PBS) for 1 h, 3 h, and 6 h. No differences were observed between NAM-treated and control-treated samples at any time point. (b) S. aureus (˜1×10⁸ CFU/mL in THB) was incubated with or without 50 mM NAM. No difference in CFU was observed at any of the time points analyzed. Data are means±s.d. This assay was performed on two independent occasions using inocula generated from three separate bacterial cultures.

FIG. 23 demonstrates, in accordance with an embodiment of the invention, NAM used in the study is endotoxin (pyrogen) free. Two separate lots (lot #1, lot #2) of NAM, used throughout the study, were tested to confirm the absence of endotoxin. The quantitative detection of bacterial endotoxin in aqueous solutions of NAM (50 mM) was determined by end-point chromogenic Limulus amebocyte lysate endochrome method. Two separate microplate assays were performed in quadruplicate measuring (a) low concentration range (0.015-0.12 EU/mL) and (b) high concentration range (0.15-1.2 EU/mL). Dashed line indicates the limit of detection. Data are means±s.d.

FIG. 24 demonstrates, in accordance with an embodiment of the invention, there is no difference in killing activity of S. aureus using whole blood from WT versus heterozygous mice.

FIG. 25 demonstrates, in accordance with an embodiment of the invention, BMDMs from WT and C/EBPε heterozygous mice infected with S. aureus Pig1 (MOI 10, and 50), followed by subsequent Western blot for CAMP, LF, C/EBPε. There are no differences between WT and HET.

FIG. 26 demonstrates, in accordance with an embodiment of the invention, confirmation of depletion of PMNs after antibody injection. FIG. 26a: WT mice (n=3) were made neutropenic by i.p. administration of 150 μl of anti-mouse PMN antibody 24 h prior (day −1) to s.c. infection with S. aureus on day 0 and every 24 h thereafter until sacrifice on day 4. Control mice (n=3) received equal amounts of normal serum. The population of PMNs (Ly6G⁺, CD11b⁺) in the spleens of mice treated with antibody versus serum (P=0.003) were analyzed by flow cytometry on day 4. FIG. 26b shows that macrophages/monocytes are not affected (depleted) by the anti-mouse PMN antibody used in this study (P>0.05).

FIG. 27 demonstrates, in accordance with an embodiment of the invention, WT mice treated with NAM (250 mg/kg/day; i.p.) for (1) 24 h, (2) 48 h, or 3 (72 h). BMMCs were subsequently removed from the mice and Western blots performed. BMMCs from n=3 mice pooled at each time point.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3d ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein:
The acronym “IP” means intraperitoneal
The acronym “KO” means knockout
The acronym “WT” means wild type
The acronym “SC” means subcutaneous
The acronym “PMA” means phorbol 12-myristate 13-acetate
The acronym “MSSA” means methicillin-sensitive Staphylococcus aureus
The acronym “MRSA” means methicillin-resistant Staphylococcus aureus
The acronym “CAMP” means cathelicidin(-related) antimicrobial peptide
The acronym “CEBPε” means CCAAT/enhancer binding protein epsilon
The acronym “SGD” means neutrophil-specific granule deficiency (SGD)
The acronym “SA” means Staphylococcus aureus
As described herein “U937 cells” means a cell line used in biomedical research that were originally isolated from the histiocytic lymphoma of a 37 year old male patient and are used to study the behavior and differentiation of monocytes. U937 cells mature and differentiate in response to a number of soluble stimuli, adopting the morphology and characteristics of mature macrophages.
The term “PMTε” means a zinc-inducible C/EBPε expression vector
The acronym “NAM” means nicotinamide
The acronym “PMN” means polymorphonuclear leukocytes
The term “immunoboosting” means boosting, enhancing, or otherwise augmenting the natural immune response.
The term “prophylactic dose” means a dose that reduces the likelihood of acquiring an infection or developing a condition.

Steady advances in molecular medicine and genetics have helped broaden the understanding of the underlying pathophysiology of leukocyte disorders and provided a clearer representation of how cells and other factors of the immune system interact. Recently, the inventors and others established the essential role of C/EBPε in the normal maturation and function of neutrophils and monocytes/macrophages¹¹⁻¹⁷. Absence of functional C/EBPε causes substantial in vitro abnormalities in these myeloid cells, including abnormal nuclear morphology, defects in stimulated oxygen metabolism, and bactericidal activity, as well as loss of all secondary (specific) granule proteins. The phenotype of C/EBPε-deficient mice closely resembles SGD in humans and resulted in the discovery of germline loss-of-function mutations involving CEBPE in individuals suffering from this disease^{8,9}.

In the present invention, the inventors substantiated the clinical finding that patients with C/EBPε deficiency are highly prone to S. aureus infection. Following S. aureus challenge, C/EBPε-deficient mice exhibited dramatic skin pathology, were unable to clear S. aureus at the infection site, and permitted systemic spread of the bacteria to the spleen and kidneys. The underlying defects of C/EBPε-deficient neutrophils are many, and include the absence of critical antimicrobial factors such as LF, and cathelicidins (e.g., CAMP), which are likely to contribute to the dramatic infection phenotype. In vivo, increased number of these phagocytic cells not only failed to compensate for the severe functional defect; paradoxically, they appear to contribute to the severity of infection since depletion of the defective neutrophils improved clearance of S. aureus and reduced the size of necrotic lesions. The inventors showed that ineffective clearance of S. aureus by C/EBPε^{-/-} neutrophils, even compared to extracellular killing mechanisms, likely permitted S. aureus to thrive within neutrophils, which further aggravated the infection.

Not unlike SGD, another human immunodeficiency condition, chronic granulomatous disease (CGD), predisposes the host to infections because the phagocytic cells from the patients are unable to generate reactive oxygen species³¹⁻³³. The standard treatment for CGD for many years consisted of IFN-γ, which has been shown to be effective in reducing the incidence and severity of infections in CGD patients³². IFN-γ is known to stimulate a number of phagocytic cell functions, including the induction of superoxide formation, upregulation of integrins and FcRγ, reduction of phagocytic vacuole pH, and degradation of intracellular tryptophan³¹⁻³³. The inventors showed that priming of C/EBPε-deficient BMDM with IFN-γ prior to infection with S. aureus effectively increased the ability of the macrophages to clear the bacteria. Moreover, systemic treatment with IFN-γ prior to and during infection, significantly improved clearance of S. aureus to a level that was comparable to untreated WT mice. Overall, the application of IFN-γ could be a practical strategy for prophylaxis of SGD patients against common infections such as S. aureus.

The profound phenotype of infected C/EBPε-deficient mice suggests that this myeloid-specific factor controls a critical antimicrobial program tailored for killing of pathogens. Low plasma levels of C/EBPε-regulated antimicrobials have been shown to be predictive of increased risk of death attributable to infection in humans³⁴. Based on the dramatic infection phenotype of the C/EBPε-deficient mice, the inventors hypothesized and demonstrated that enhanced transcriptional activity of C/EBPε in normal subjects, either by induced overexpression or by induction with NAM, could have a significant and often dramatic effect on killing of S. aureus both in vitro and in vivo. The absence of improved bacterial clearance in our C/EBPε-deficient models further underlines the significant interplay between C/EBPε and NAM.

HDAC inhibitors, such as NAM, influence transcriptional expression by controlling chromatin condensation, and regulate proteins involved in acetylation¹⁹⁻²³. As a precursor to NAD+, NAM can block deacetylation and the regeneration of NAD+ through interception of an ADP-ribosyl-enzyme-acetyl peptide intermediate^{25-27,35}. NAD+-dependent transcriptional regulation was previously demonstrated for the highly conserved family members, C/EBPα and C/EBPβ³⁶. Moreover, transcriptional activity mediated by C/EBPβ can be enhanced by increased acetylation of its lysine residues by the HDAC inhibitors NAM and trichostatin¹⁸. While not wishing to be bound by any one particular theory, in line with these findings, it appears that NAM, in its role as an epigenetic modulator, can increase the transcriptional activity of a broad number of downstream targets mediated by C/EBPε, including the well-recognized antimicrobials CAMP and LF^{9,15,37}.

The next issue for the inventors to determine was whether the therapeutic effect documented with S. aureus could be achieved in human subjects using safe NAM doses. In human trials, NAM is frequently administered as a modifier to patients undergoing radiotherapy^{38,30,49,50}. In these trials, a plasma concentration of 1 mM NAM is routinely achieved, a concentration that the inventors used in their peripheral whole blood killing assays to demonstrate NAM efficacy. Therefore, a NAM concentration safely achievable in humans could provide protection against S. aureus infection.

The inventors' finding that NAM has a dramatic effect on immune-mediated killing of S. aureus in mice and in humans has a number of therapeutic implications. In an age when the number of antibiotics in the pipeline is limited and development of resistance occurs rapidly, use of complementary strategies to antibiotic treatment would provide a method of limiting development of antibiotic resistance. Further, because C/EBPε controls the transcription of a number of important antimicrobial factors, induction of resistance to multiple host factors is less likely. Likewise, the use of an immune boosting strategy coupled with conventional antibiotics is likely to provide important synergy.

While the use of NAM against conventional bacteria has not been previously reported, the vitamin compound has shown promising efficacy in treatment of Mycobacterium tuberculosis infection based on human studies performed in the 1960s⁴⁰. Similarly, NAM administration is associated with a beneficial effect in patients with HIV infection⁴⁰. Immune response to HIV is hypothesized to cause vitamin B3 deficiency, and the benefit of nicotinamide supplementation comes from correction of this deficiency. In both cases, the role of C/EBPε in combating HIV and M. tuberculosis is unknown. Recently, Wurtele et al. reported that modulation of the yeast histone H3 Lys56 by NAM sensitized Candida albicans for genotoxic and antifungal agents⁵¹. The inventors have demonstrated herein that NAM, as an HDAC-inhibitor, can improve host defense and thereby promote bacterial clearance. As disclosed herein, NAM is not only effective against S. aureus, it also has demonstrated efficacy against other major human pathogens such as K. pneumoniae and P. aeruginosa in the human peripheral blood killing assay, and therefore NAM is likely to be effective against a number of other pathogens as well. These findings reflect the broad potential of compounds with the ability to stimulate the activity of C/EBPε and related antimicrobial targets.

In summary, the inventors have demonstrated that C/EBPε is a regulatory factor that significantly impacts the host's ability to fight S. aureus infections. Manipulation of C/EBPε expression in neutrophils and monocytes/macrophages, either by forced overexpression or by pharmacological application of NAM, leads to a clear therapeutic effect on S. aureus infection. The inventors' results indicate that compounds exerting modulatory effects on the myeloid-specific transcription factor C/EBPε may be useful as antimicrobial therapeutics.

The present invention is based, at least in part, on these findings as well as others further described herein.

In certain embodiments, the present invention provides a method for treating an infection caused by a pathogen, by providing a composition that upregulates the expression of CCAAT/enhancer binding protein epsilon (C/EBPε), and administering a therapeutic dose of the composition to an individual having an infection caused by a pathogen, whereby an enhanced immune response to the infection results in the individual.

In certain embodiments, the composition acts as an activator/agonist of C/EBPε. In some embodiments, the composition includes a histone-deacetylase (HDAC) inhibitor. In certain embodiments, the composition includes an HDAC class III inhibitor. In certain embodiments, the composition is vitamin B3 or an analog, derivative or salt thereof. In certain embodiments the vitamin B3 or an analog, derivative or salt thereof is administered orally to an individual in need thereof. In certain embodiments, the route of administration may be intravenous, intramuscular or inhaled. In certain embodiments of the invention, the therapeutic dose of vitamin B3, or an analog, derivative or salt thereof is between 5 mg/kg/day to 1000 mg/kg/day. In certain embodiments, the dose is taken 1-10 times per day. In certain embodiments, the dose is administered 3-7 times per day. In certain embodiments, the dose is administered 1-2 times per day. In certain embodiments, the course of treatment is 1-20 days. In other embodiments the course of treatment is 3-15 days. In another embodiment the course of treatment is 3-10 days. In certain embodiments, the subject is a mammal, in certain embodiments, the individual is a human. In certain embodiments, the infection is caused by one or more pathogens, including: parasites, fungi, bacteria, viruses, or combinations thereof. In certain embodiments, the infection is caused by a pathogen including: Staphylococcus aureus (S. aureus), methicillin-resistant S. aureus, Vancomycin resistant Enterococcus, C. difficile, B. cepacia, influenza, rhinovirus, Epstein-Barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof.

As described herein, the present invention also provides a method for reducing the likelihood of acquiring or developing an infection caused by a pathogen, by providing a prophylactic dose of a composition that upregulates the expression of CCAAT/enhancer binding protein epsilon (C/EBPε); and administering the prophylactic dose to an individual in need thereof. As used herein, a “prophylactic dose” is a dose that reduces the likelihood of acquiring an infection. In certain embodiments, the composition acts as an activator/agonist of C/EBPε. In certain embodiments, the composition includes a histone-deacetylase (HDAC) inhibitor. In certain embodiments, the composition includes an HDAC class III inhibitor. In some embodiments, the composition is vitamin B3 or an analog, derivative or salt thereof. In certain embodiments, the vitamin B3 or an analog, derivative or salt thereof can be administered orally. In certain embodiments, the routes of administration include intravenous, intramuscular or inhaled. In certain embodiments, the prophylactic dose is between 5 mg/kg/day to 1000 mg/kg/day. In certain embodiments, the vitamin B3 or an analog, derivative or salt thereof is administered 1-2 times per day. In other embodiments the dose is administered every day. In still other embodiments, the prophylactic dose is administered every two days. In yet other embodiments, the prophylactic dose is administered once a week. In certain embodiments, the individual is a mammal. In certain embodiments, the individual is a human. In certain embodiments the composition has a prophylactic effect against pathogens, including: parasites, fungi, bacteria, viruses, or combinations thereof. In certain embodiments the prophylactic effect is against a pathogens including Staphylococcus aureus (S. aureus), methicillin-resistant S. aureus, Vancomycin resistant Enterococcus, C. difficile, B. cepacia, influenza, rhino virus, Epstein-Barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof.

In certain embodiments, vitamin B3 or an analog, derivative or salt thereof is used as part of a parenteral nutrition regimen. In certain embodiments the parenteral nutrition is administered to neonates or patients in the hospital that cannot eat on their own. In certain embodiments, incorporating vitamin B3 or an analog, derivative or salt thereof as part of a parenteral nutrition regimen has a prophylactic effect against pathogens including: parasites, fungi, bacteria, viruses, or combinations thereof. In certain embodiments, the prophylactic effect is against pathogens including: Staphylococcus aureus (S. aureus), methicillin-resistant S. aureus, Vancomycin resistant Enterococcus, C. difficile, B. cepacia, influenza, rhinovirus. Epstein barr virus, cytomegalovirus, adenovirus, parainfluenza virus, rotavirus, candida, ESBL gram negative pathogens, S. epidermidis, Pseudomonas. Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof.

As described herein, the present invention also provides a method of increasing an anti-inflammatory response in an individual, by providing a composition that upregulates the expression of CCAAT/enhancer binding protein epsilon (C/EBPε), and administering a therapeutic dose of the composition to an individual having an inflammatory condition. In certain embodiments, the composition is Vitamin B3 or an analog, derivative or salt thereof. In certain embodiments, the upregulation of C/EBPEε increases interleukin 10 (IL-10) function. In certain embodiments, the increased IL-10 function results in anti-inflammatory mediation of an inflammatory and/or autoimmune condition including: atherosclerosis, inflammatory bowel diseases, multiple sclerosis, rheumatoid arthritis, asthma, bacterial sepsis, Kawasaki's disease, atopic dermatitis, and other rheumatologic conditions.

In certain embodiments, the present invention provides a method for treating an infection in an individual with a defective immune response against an infection caused by a pathogen, by providing interferon-gamma, and administering a therapeutic dose of interferon-gamma to an individual having an infection. In certain embodiments, the individual is a mammal. In certain embodiments, the individual is a human. In certain embodiments, the individual has neutrophil-specific granule deficiency (SGD). In certain embodiments, the therapeutic dose is between 20-150 micrograms/m². In certain embodiments, the therapeutic dose is between 30-120 micrograms/m². In certain embodiments the therapeutic dose is between 50-100 micrograms/m². In certain embodiments, the therapeutic dose is administered daily. In certain embodiments the therapeutic dose is administered bi-weekly. In yet another embodiment, the therapeutic dose is administered 3 times per week. In certain embodiments, the infection is caused by one or more pathogens, including: parasites, fungi, bacteria, viruses, or combinations thereof. In certain embodiments, the infection is caused by one or more pathogens, including: Staphylococcus aureus (S. aureus), methicillin-resistant S. aureus, Vancomycin resistant Enterococcus, C. difficile, B. cepacia, influenza, rhinovirus, Epstein barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof.

As described herein, the present invention also provides a method of reducing the likelihood of acquiring or developing an infection caused by a pathogen by providing a prophylactic dose of interferon-gamma, and administering a prophylactic dose of interferon-gamma to an individual in need thereof. In certain embodiments, the individual is a mammal. In certain embodiments, the individual is a human. In certain embodiments, the individual has neutrophil-specific granule deficiency SGD. In certain embodiments, the prophylactic dose is between 20-150 micrograms/m². In certain embodiments, the prophylactic dose is between 30-120 micrograms/m². In yet another embodiment the prophylactic dose is between 50-100 micrograms/m². In one embodiment, the prophylactic dose is administered daily. In another embodiment the prophylactic dose is administered bi-weekly. In yet another embodiment, the prophylactic is administered 3 times per week. In another embodiment the interferon-gamma has a prophylactic effect against pathogens, including: parasites, fungi, bacteria, viruses, or combinations thereof. In certain embodiments, the prophylactic effect is against pathogens, including: Staphylococcus aureus (S. aureus), methicillin-resistant S. aureus, Vancomycin resistant Enterococcus, C. difficile, B. cepacia, influenza, rhinovirus, Epstein barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof.

In various embodiments, the vitamin B3 or interferon-gamma may be provided as pharmaceutical compositions including a pharmaceutically acceptable excipient along with a therapeutically effective amount of the vitamin B3 and/or interferon-gamma. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl mnonostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed, 20th edition, Williams & Wilkins PA, USA) (2000).

Typical dosages of an effective amount of the vitamin B3 or interferon-gamma can be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method.

The present invention is also directed to a kit to treat and/or prevent a pathogenic infection in a mammal in need thereof. The kit is useful for practicing the inventive method of treating and/or preventing a pathogenic infection, in particular an infection caused by a pathogen selected from the group consisting of: parasites, fungi, bacteria, viruses, or combinations thereof. The infection may also be caused by Staphylococcus aureus (S. aureus), methicillin-resistant S. aureus, Vancomycin resistant Enterococcus, C. difficile, B. cepacia, influenza, rhinovirus, Epstein barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof. The kit is an assemblage of materials or components, including at least one of the inventive compositions. Thus, in some embodiments the kit contains a composition including vitamin B3 or interferon-gamma as described above.

The exact nature of the components configured in the inventive kit depends on its intended purpose. For example, some embodiments are configured for the purpose of treating a pathogenic infection. Other embodiments are configured for prophylaxis. Yet other embodiments are for the purpose of treating inflammation. In one embodiment, the kit is configured particularly for the purpose of treating mammalian subjects. In another embodiment, the kit is configured particularly for the purpose of treating human subjects. In another embodiment, the kit is configured for treating adolescent, child, or infant human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat and/or prevent an infection caused by a pathogen. Instructions for use may include instructions to administer a dose of vitamin B3 2 times per day. Instructions for use may include instructions to administer a dose of interferon-gamma from 1-10 times per week. Particularly, instructions for use may include instructions to administer 5 mg/kg/day to 1000 mg/kg/day of vitamin B3 via one or two doses per day. Instructions for use may include instructions to administer 50-100 micrograms of interferon-gamma 1-10 times per week. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in chemotherapy. As used herein, the term “package” refers to a suitable solid matrix or material such as glass, plastic, paper, foil, and the like, capable of holding the individual kit components. Thus, for example, a package can be one or more glass vials or plastic containers used to contain suitable quantities of an inventive composition containing vitamin B3 or interferon-gamma. The packaging material generally has an external label which indicates the contents and/or purpose of the kit and/or its components.

### EXAMPLES

### Example 1

### Impaired Response of C/EBPε^{-/-} mice to S. aureus Infection

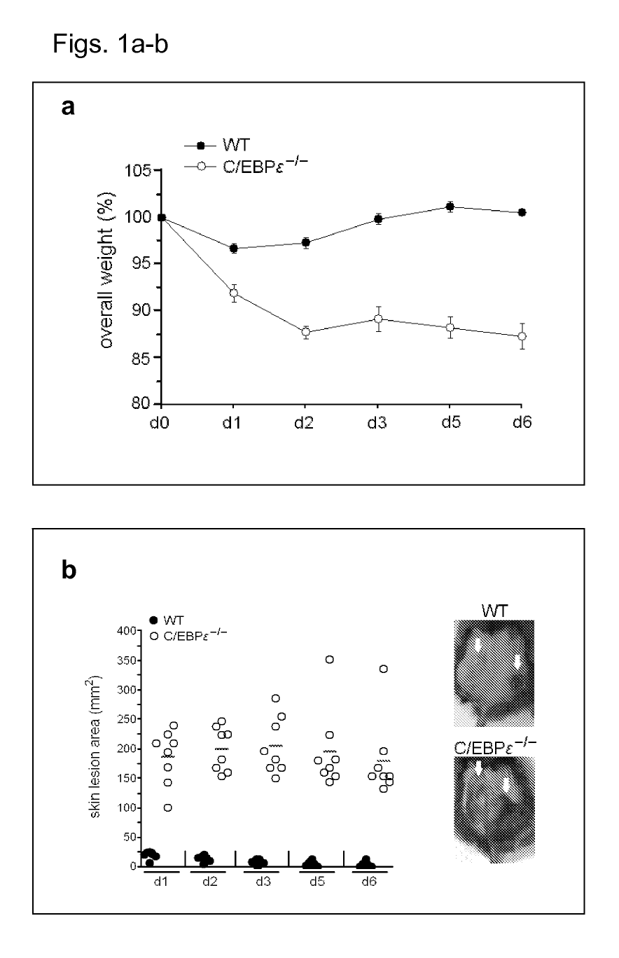

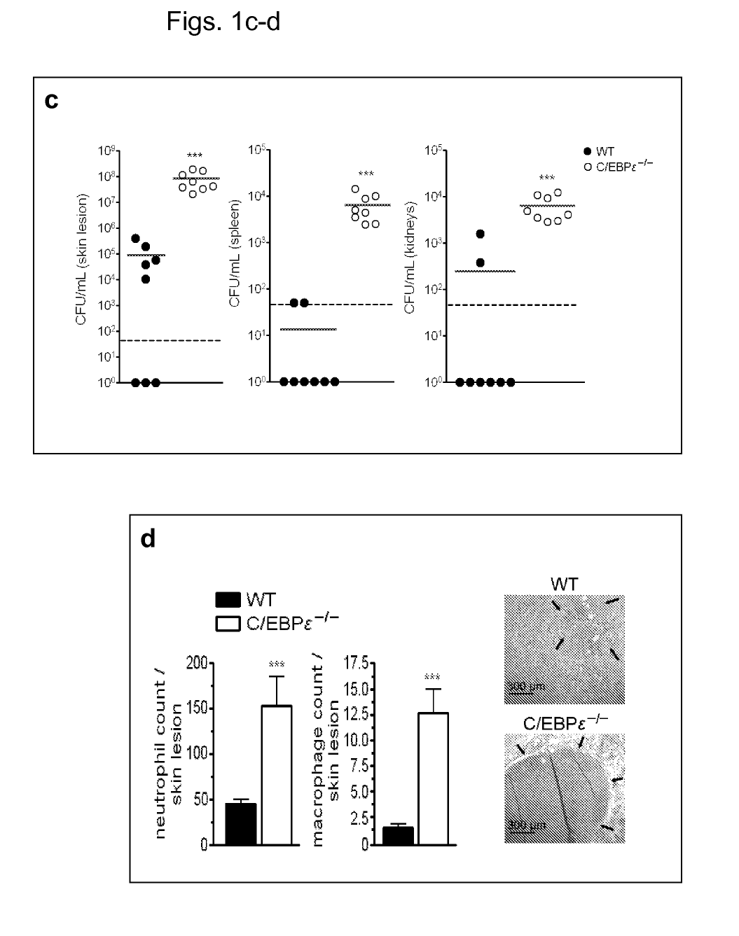

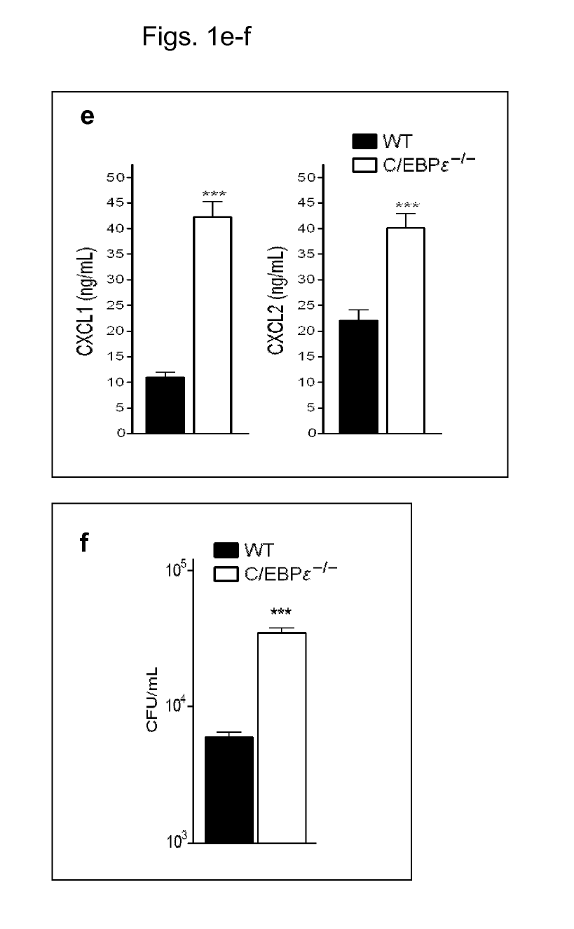

Humans and mice without functional C/EBPε have significant neutrophil and monocyte/macrophage defects, comparable to individuals with SGD^{7-9,11-17}. To determine the critical role of C/EBPε in S. aureus infection, the inventors challenged wild-type (WT) and C/EBPε^{-/-} mice with different doses of S. aureus subcutaneously. Significantly, compared to WT mice, C/EBPε^{-/-} mice exhibited dramatic weight loss, larger skin lesion size, and higher CFU in the lesions (FIG. 1a-c). Additionally, subcutaneous (s.c.) infection of C/EBPε^{-/-} mice was associated with increased systemic spread of bacteria to the spleen and kidneys on day 6 post infection (p.i.) (FIG. 1c).

Histopathological evaluation with hematoxylin and eosin (H&E) revealed a significantly larger number of neutrophils and macrophages in skin lesions of C/EBPε^{-/-} mice 24 h after infection compared to WT mice (FIG. 1d) which was accompanied by high levels of the chemokines CXCL1 and CXCL2 (FIG. 1e). These data suggest that enhanced accumulation of phagocytic cells at the infection site in C/EBPε^{-/-} mice failed to control S. aureus infection and point to the severe antimicrobial defect in C/EBPε^{-/-} phagocytic cells.

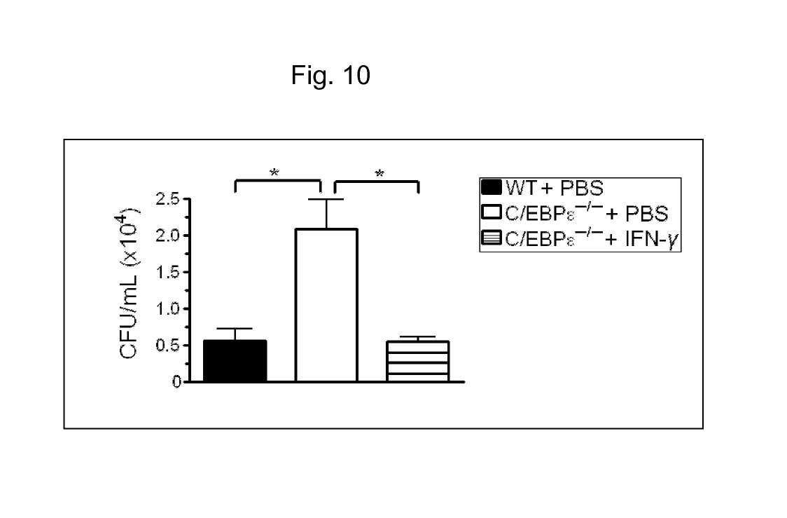

To formally assess the ability of C/EBPε^{-/-} phagocytic cells to kill S. aureus, the inventors employed a well described phagocytic survival assay in which peripheral blood from WT and knockout mice was infected with S. aureus ex vivo. Bacterial clearance was significantly decreased in the blood of the C/EBPε^{-/-} mice compared to WT (FIG. 1f). Reduced bacterial clearance by C/EBPε^{-/-} bone-marrow derived macrophages (BMDM) was also observed (FIG. 10).

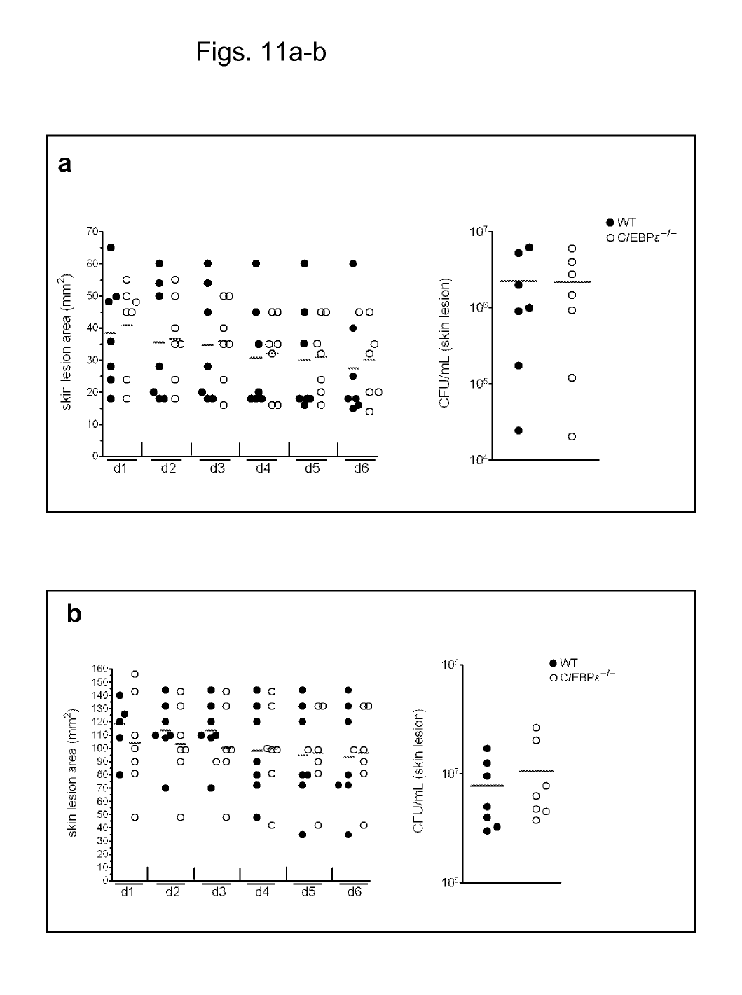

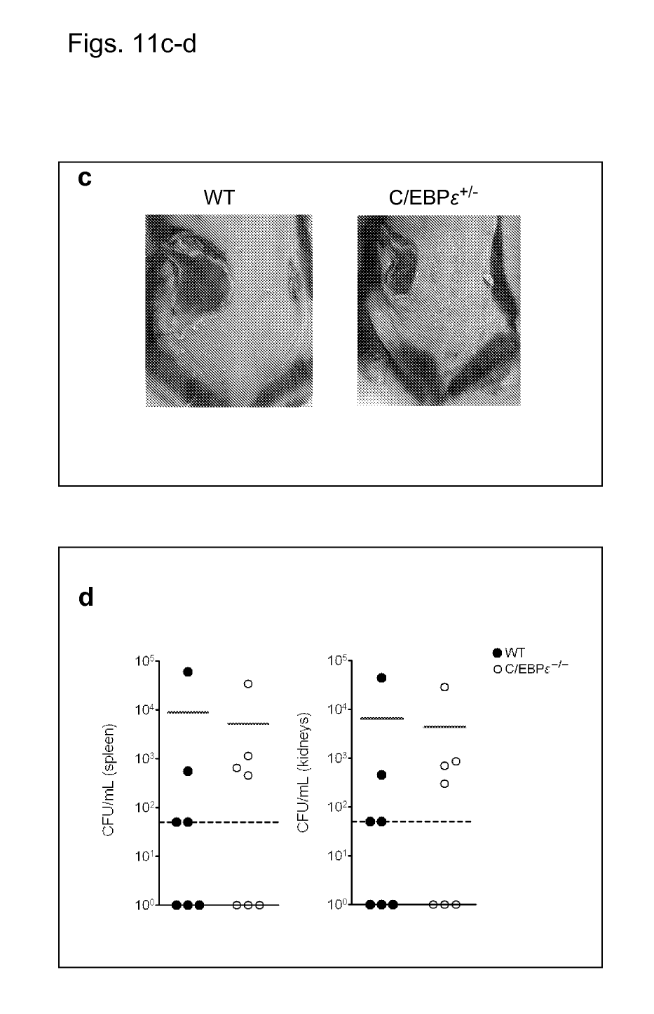

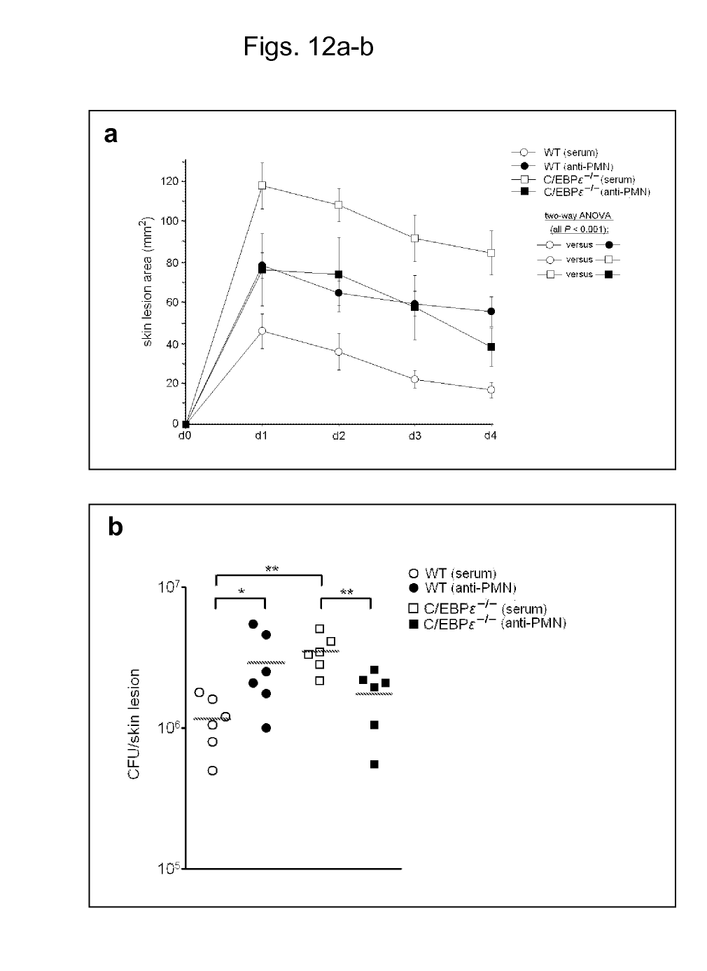

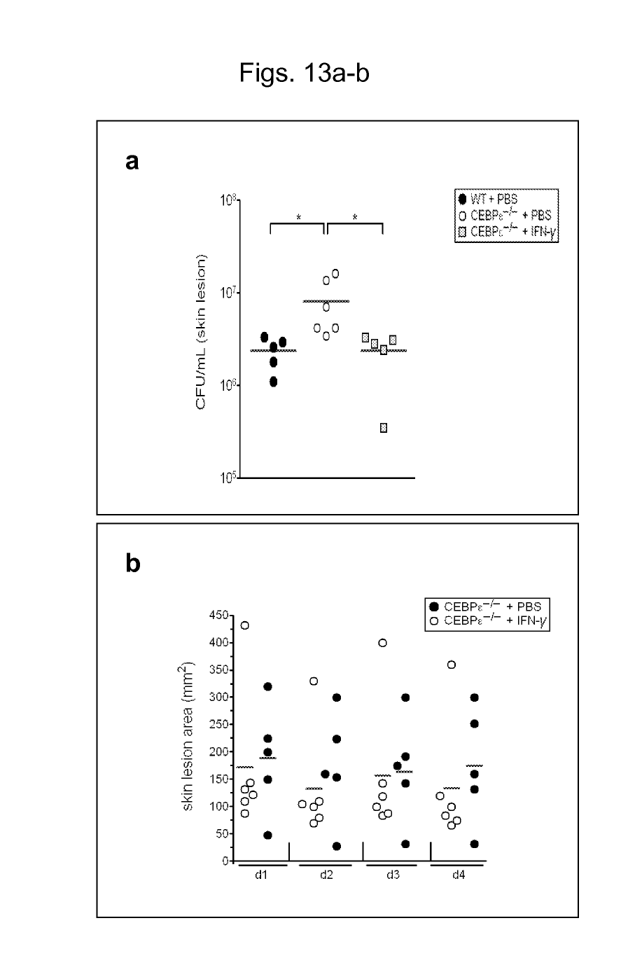

Mice heterozygous for C/EBPε, unlike C/EBPε^{-/-} mice, presented no aberrant in vivo response during infection with S. aureus (FIG. 11), suggesting that one allele of C/EBPε is sufficient for adequate immunity. Taken together, the inventors' findings underline the importance of C/EBPε in host defense against S. aureus.

### Example 2

### Depletion of Neutrophils in C/EBPε^{-/-} Mice Ameliorates Clearance of S. Aureus

Neutrophils are an important component of host immune response against S. aureus. To determine the contribution of C/EBPε^{-/-} neutrophils towards infection in vivo, the inventors performed infection experiments in WT and C/EBPε^{-/-} mice depleted of neutrophils. Depletion was achieved by daily injection of mice with a mouse anti-polymorphonuclear neutrophil (PMN) antibody starting 24 h prior to s.c. infection with S. aureus.

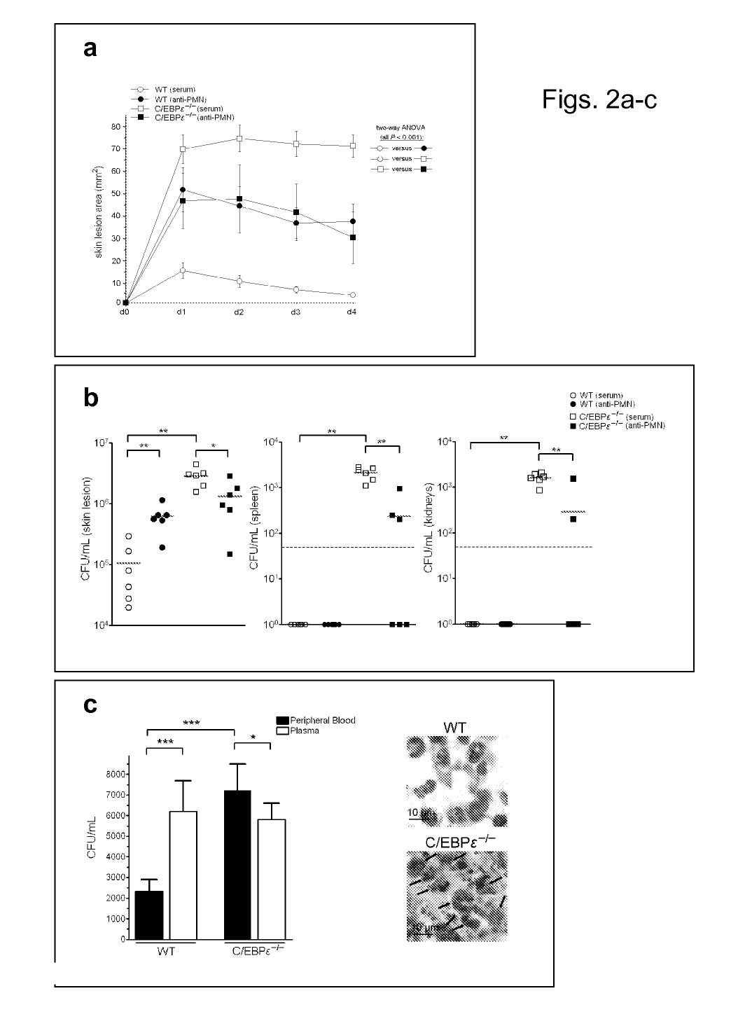

In the absence of neutrophils, WT mice showed larger skin lesions and higher CFU in the skin (FIG. 2a,b). By contrast, C/EBPε^{-/-} mice depleted of neutrophils showed significantly smaller skin lesions, fewer CFU within the lesion, and reduced systemic spread of bacteria to the spleen and kidneys (FIG. 2a,b) suggesting that C/EBPε^{-/-} neutrophils not only clear bacteria poorly, but they could exacerbate the infection. Depletion of neutrophils resulted in comparable lesion sizes and CFU recovery in infected WT and C/EBPε^{-/-} mice, which once again points to neutrophils from C/EBPε^{-/-} mice as the major contributor to infection severity in those knockout mice.

To investigate how the neutrophils from C/EBPε^{-/-} mice paradoxically promoted survival of S. aureus during infection, the inventors hypothesized that those defective neutrophils kill S. aureus even less effectively compared to host extracellular killing mechanisms. Therefore, neutrophils only serve to hinder extracellular clearance of S. aureus in C/EBPε^{-/-} mice. First, the inventors compared killing of S. aureus by cell-free plasma (to simulate extracellular killing) and peripheral whole blood isolated from WT and C/EBPε^{-/-} mice. As shown in FIG. 2c, whole blood from WT mice cleared S. aureus significantly better than plasma from WT mice. By contrast, whole blood from C/EBPε^{-/-} mice was less effective at killing S. aureus compared to C/EBPε plasma. In murine infection, H&E staining of infected skin showed C/EBPε^{-/-} neutrophils that are full of intracellular S. aureus and with few extracellular bacteria. By contrast, only few intracellular and extracellular bacteria were found at the lesion site of WT mice.

While not wishing to be bound by any one particular theory, taken together, these data suggest that neutrophils from C/EBPε^{-/-} mice do not provide an antimicrobial benefit to the host and even contribute to more severe infection.

### Example 3

### IF-γ can Compensate for the Impaired Immune Response in C/EBPε^{-/-} Mice

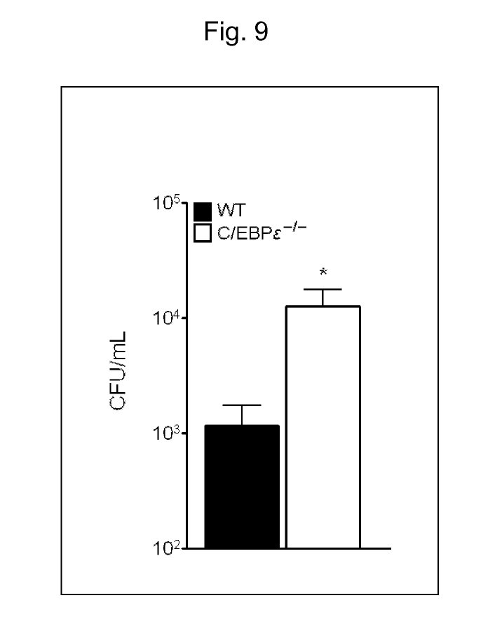

To address whether therapeutic immunostimulation can improve the outcome of bacterial infection in C/EBPε^{-/-} mice, the inventors tested the effect of the robust immunomodulator IFN-γ. BMDM from C/EBPε^{-/-} mice, treated with IFN-γ for 48 h prior to infection, showed improved killing of S. aureus, to a level comparable to PBS-treated WT BMDM (FIG. 9).

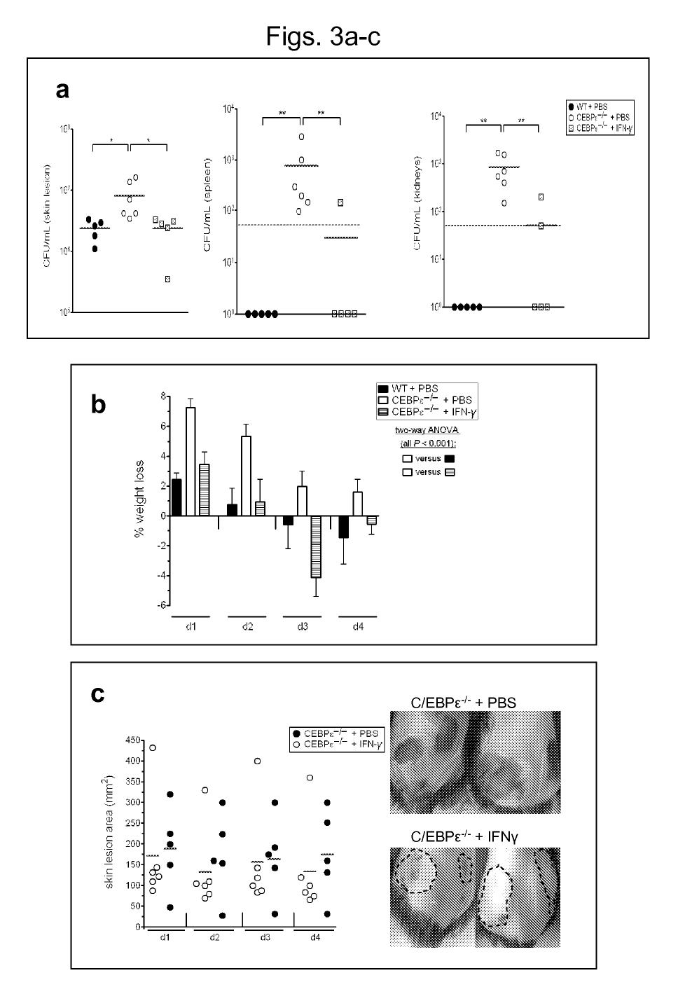

Next, IFN-γ (5,000 U) was administered to C/EBPε^{-/-} mice daily for 4 days, and infected the mice with S. aureus s.c. 48 h after the first dose of IFN-γ. C/EBPε^{-/-} mice treated with IFN-γ showed significantly lower CFU in skin lesions, spleen, and kidneys, compared to PBS-treated C/EBPε^{-/-} mice (FIG. 3a). Importantly, comparable numbers of bacteria were found in the skin and inner organs of IFN-γ-treated C/EBPε^{-/-} mice and PBS-treated WT mice (FIG. 3a). Changes in the body weight were also similar between the IFN-γ treated C/EBPε^{-/-} mice and WT control (FIG. 3b). Interestingly, while IFN-γ dramatically ameliorated the area of dermonecrosis in C/EBPε^{-/-} mice, it had no effect on overall lesion size (FIG. 3c).

Overall, the systemic application of recombinant IFN-γ helped compensate for the defective innate immune system in C/EBPε^{-/-} mice.

### Example 4

### Induced Overexpression of C/EBPε Promotes Macrophage Killing of S. Aureus In Vitro

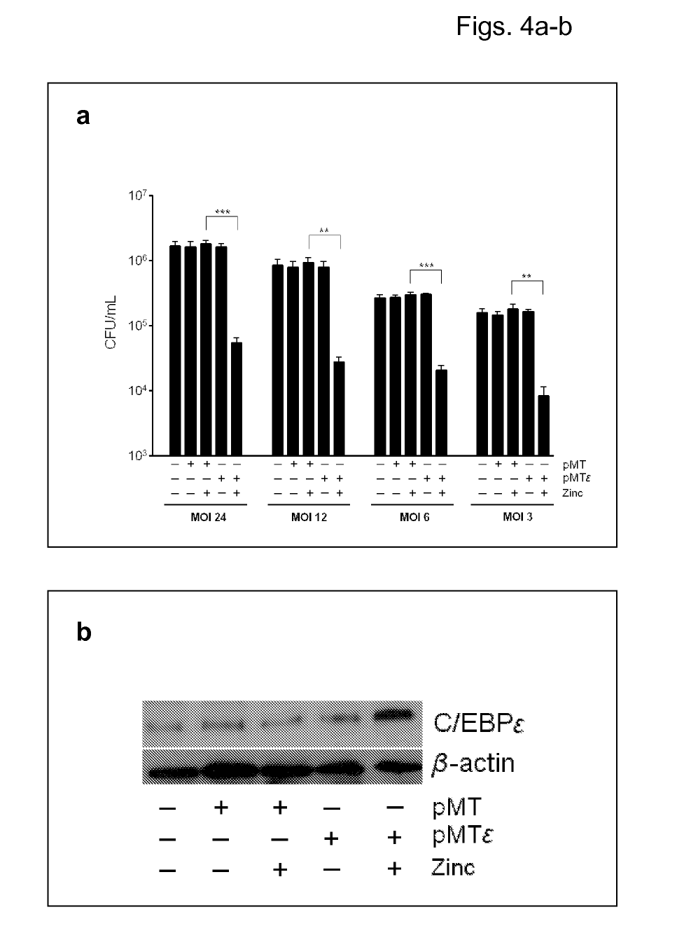

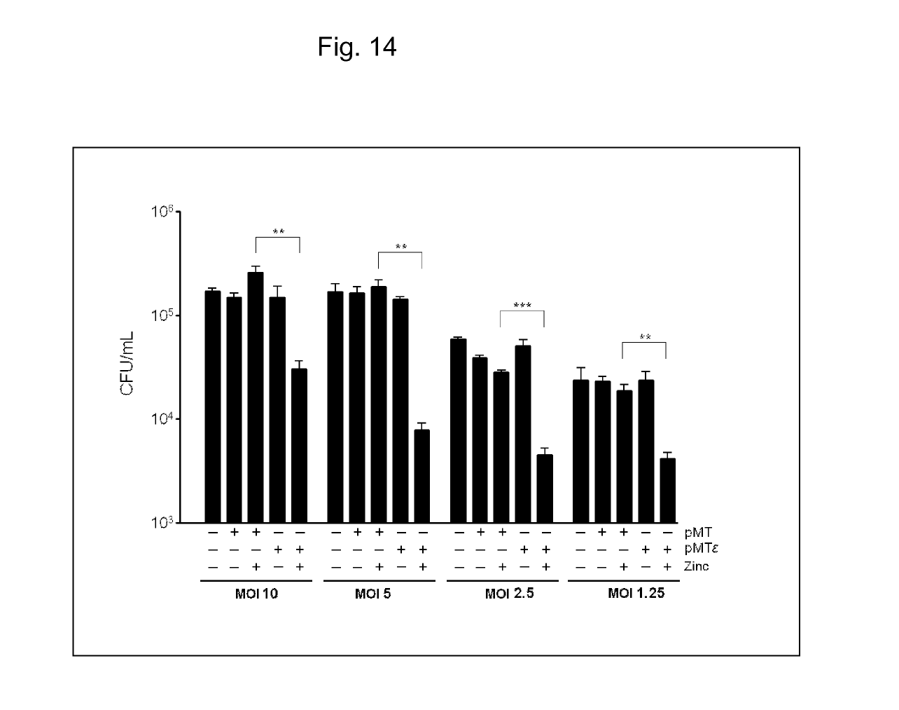

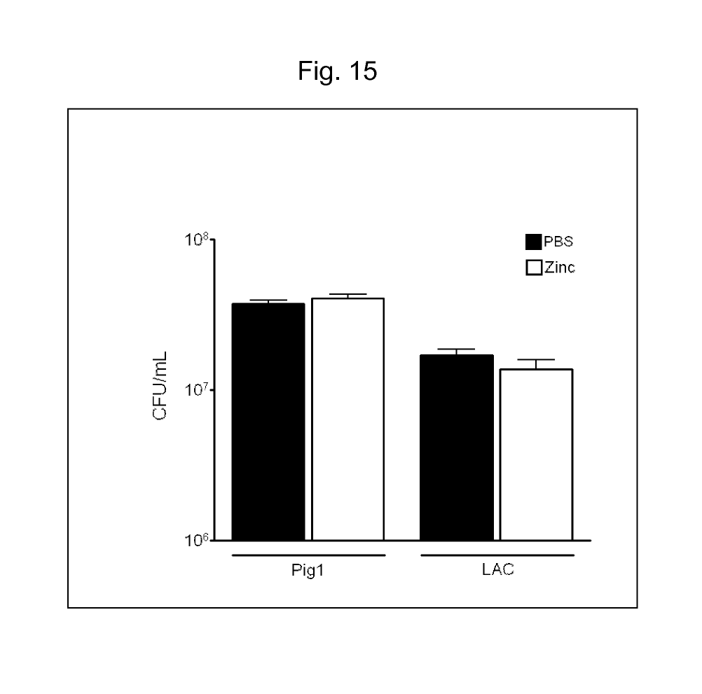

Because C/EBPε plays a role in the host immune response against S. aureus infection, the inventors hypothesized that increased expression of C/EBPε could enhance immune killing of bacteria. The inventors investigated this possibility by inducing overexpression of C/EBPε in U937, a pro-monocytic cell line that has routinely been used to study human macrophage effector functions^{28,29}. U937 cells were stably transfected with a zinc-inducible C/EBPε-expression vector (pMTε) and differentiated to macrophages using phorbol 12-myristate 13-acetate (PMA). Interestingly, the PMA-derived macrophages with forced expression of C/EBPε killed up to 1.5 log₁₀ CFU/mL more S. aureus compared to vector control (FIG. 4a). The inventors were able to repeat these findings using a strain of CA-MRSA (FIG. 14). Zinc alone had no effect on the viability and growth of S. aureus (FIG. 15).

### Example 5

### NAM Increases Activity of C/EBPε in Myeloid Cells Both In Vitro and In Vivo

Based on the above findings, the inventors next asked whether a pharmacologic agent could induce overexpression of C/EBPε to promote more effective immune-mediated killing of S. aureus. Though the regulation of C/EBPε expression is not known, Skokowa and colleagues recently demonstrated that NAM, a well-established HDAC inhibitor, could epigenetically modify and enhance expression of C/EBPα and C/EBPβ³⁶. The inventors tested first the ability of NAM, a well-established HDAC inhibitor, to modify acetylation of CEBPE and promote enhanced expression of C/EBPε.

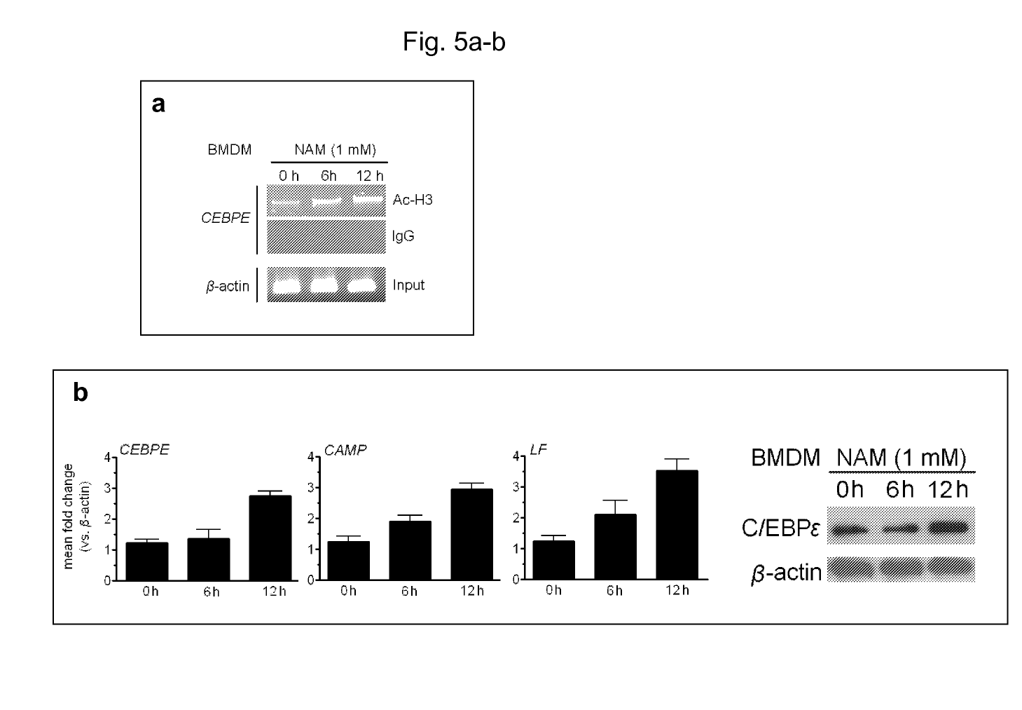

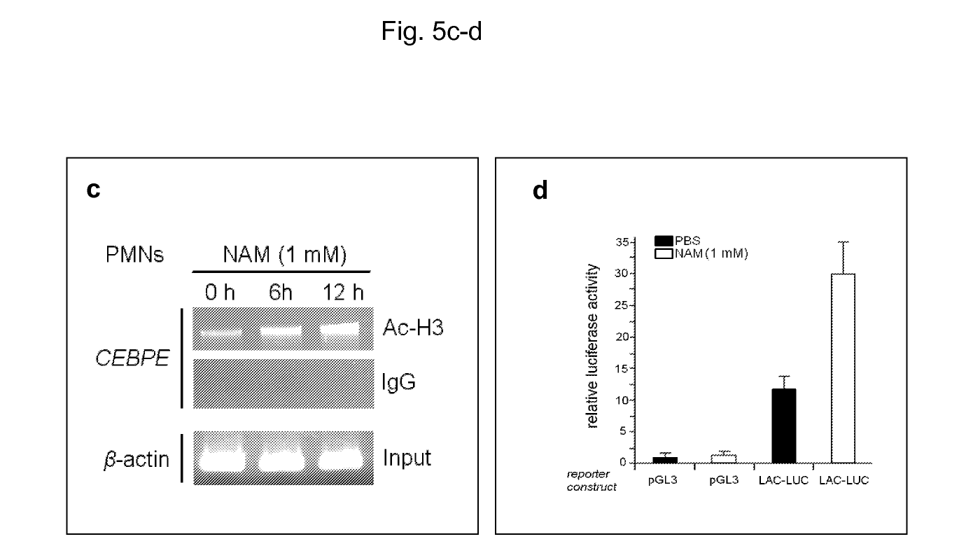

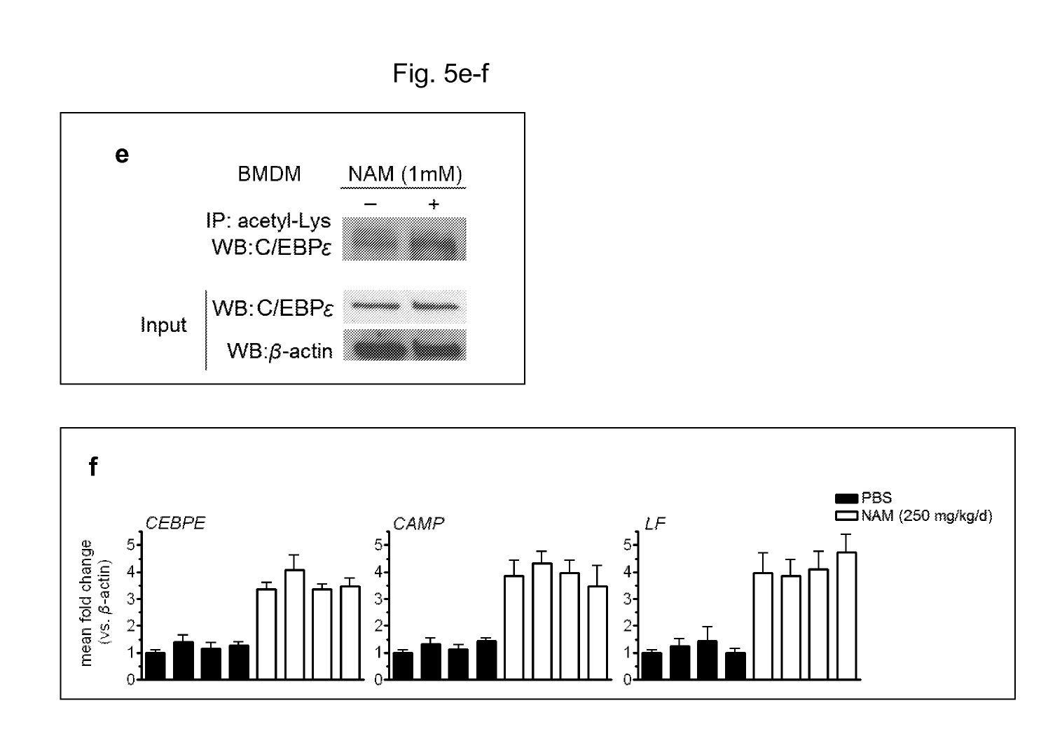

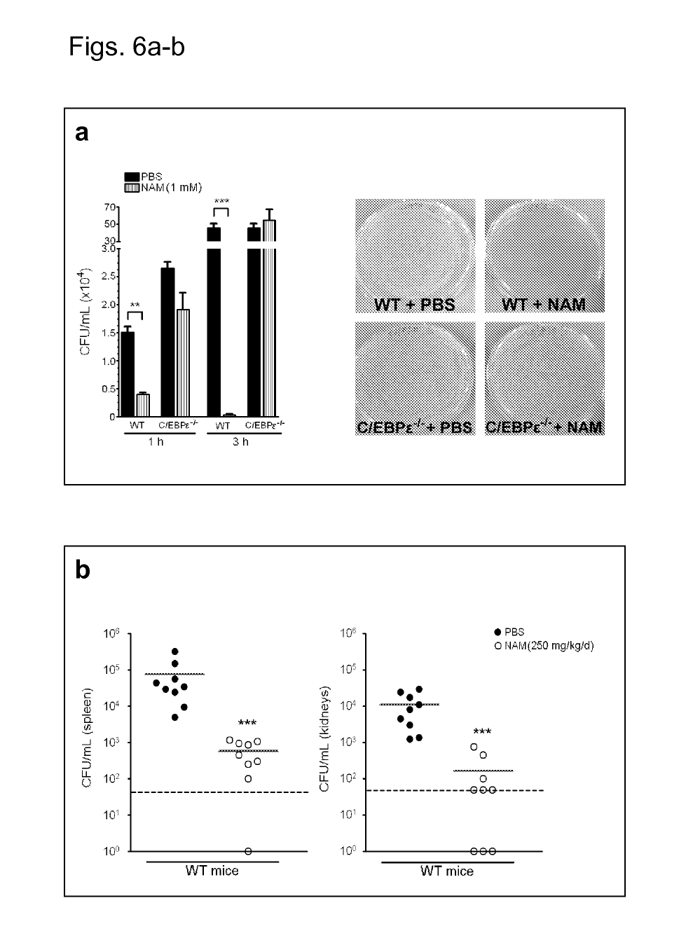

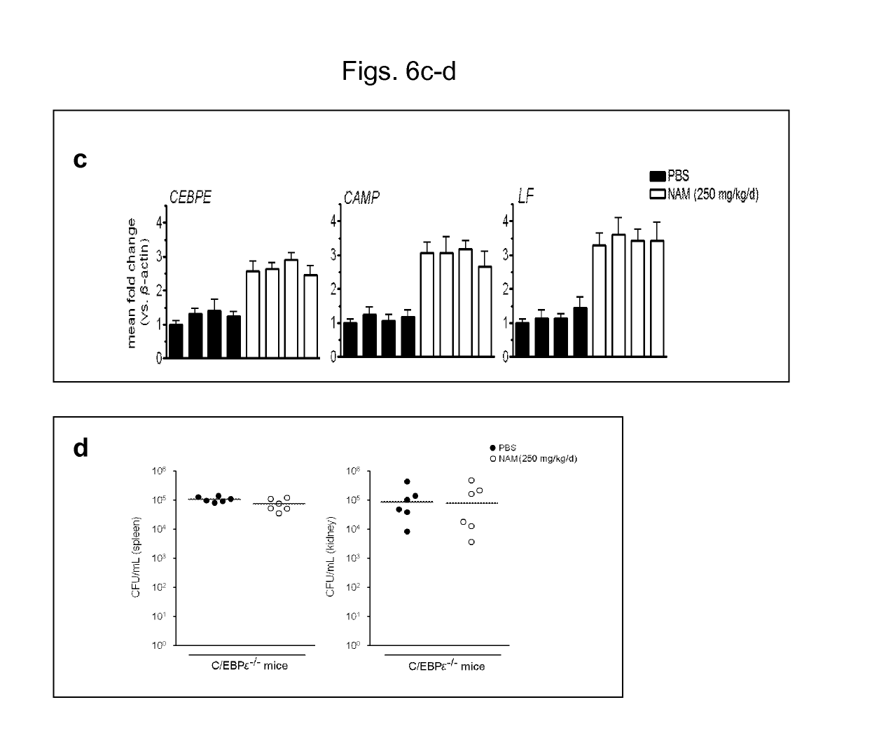

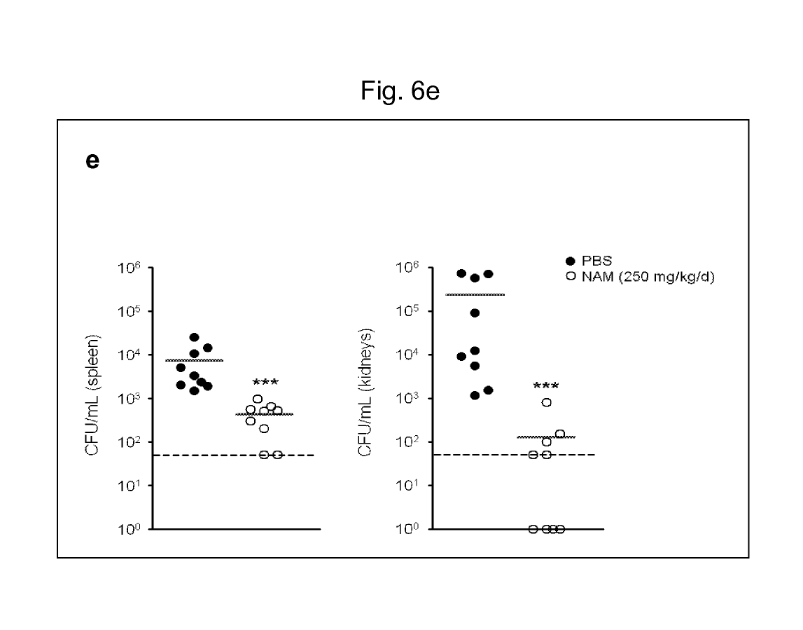

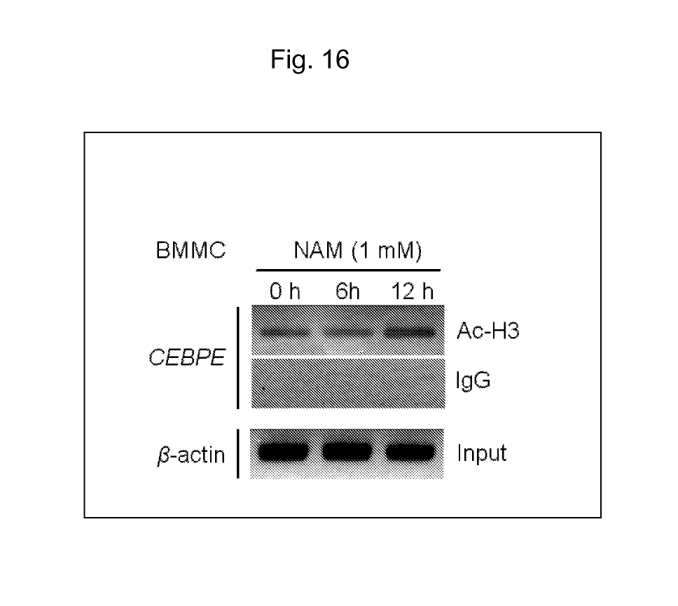

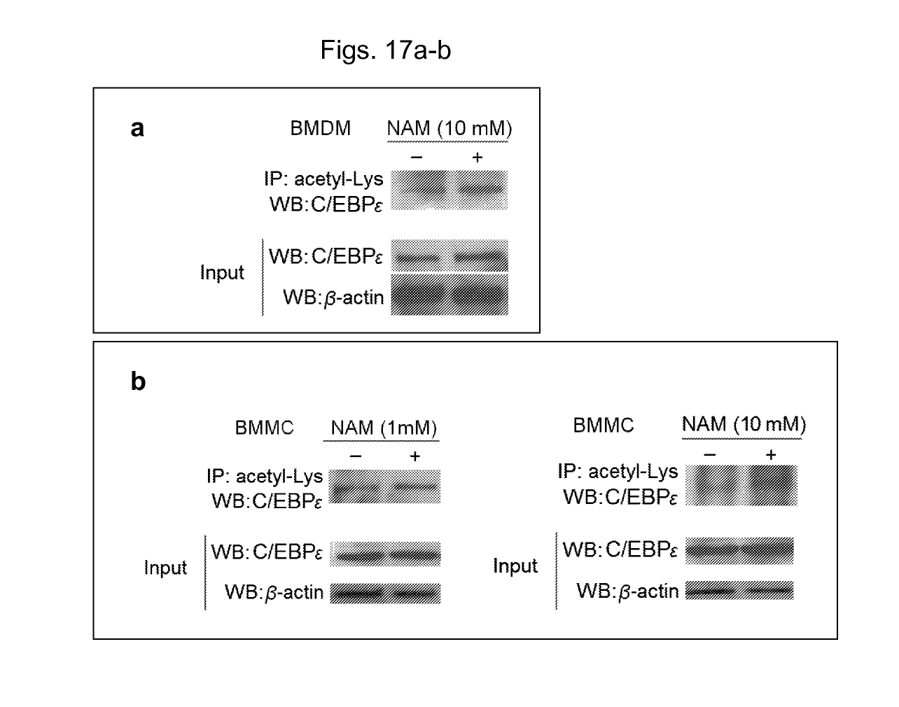

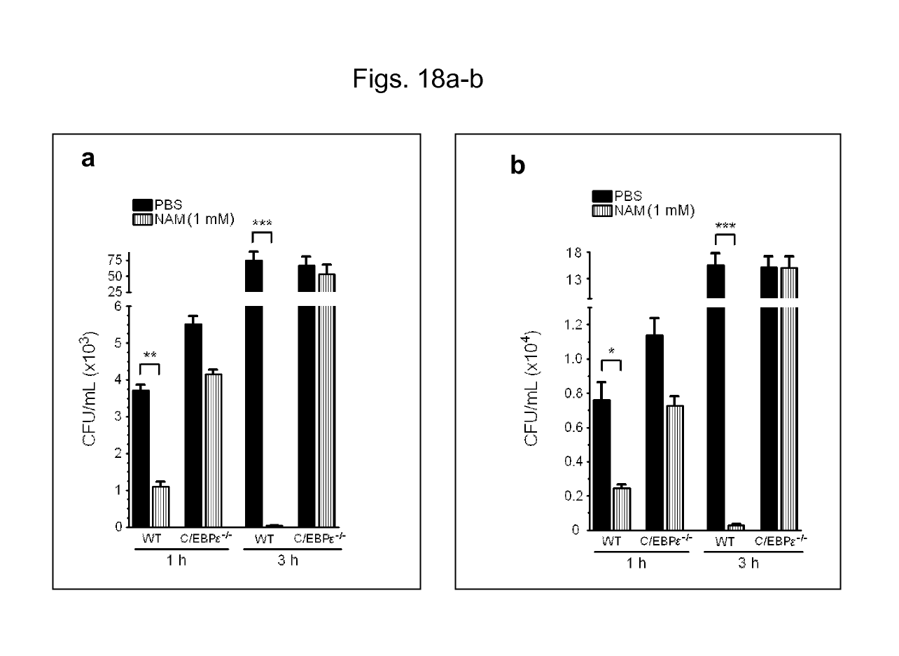

Upon exposing WT BMDM to NAM (1 mM) for 6-12 h, the inventors detected a 5-fold increase in the level of lysine acetylation on histone H3 at the promoter region of the CEBPE (FIG. 5a). NAM treatment of BMDM also resulted in elevated mRNA and protein levels of C/EBPε, and increased expression of downstream antimicrobial targets cathelicidin(-related) antimicrobial peptide (CAMP) and LF (FIG. 5b). Treatment of murine bone marrow mononuclear cells (BMMC) and human PMNs with NAM induced a similar increase in histone acetylation specifically at the CEBPE promoter site (FIG. 16 and FIG. 6c).

To confirm the regulatory effect of NAM on the transcriptional activity of C/EBPε, the inventors fused the proximal promoter fragment (−230 to +39) of LF including a putative C/EBP-binding site to a luciferase reporter construct. Following transient transfection of U937 pro-monocytic cells with this construct, treatment with 1 mM NAM resulted in a 2.5-fold increase in LF reporter gene activity compared to PBS control (FIG. 5d).

The inventors also investigated the influence of NAM on the acetylation of C/EBPε protein in BMMC and BMDM derived from WT mice. After 6 h of treatment with NAM, acetylation of lysine residues of C/EBPε was 2- to 4-fold higher as measured by immunoprecipitation, suggesting increased protein activity of C/EBPε in myeloid cells (FIG. 5e).

To confirm the inventors' in vitro findings, NAM was administered systemically to non-infected WT mice (250 mg/kg/day i.p.), and expression of CEBPE and downstream factors within BMMC were measured. Expression analysis in these cells after 72 h revealed a 3- to 4.5-fold higher mRNA levels of CEBPE as well as CAMP and LF, compared to PBS-treated mice (FIG. 5f).

### Example 6

### NAM Enhances Killing of S. Aureus in Mice and in Human Blood by a C/EBPε-Dependent Mechanism

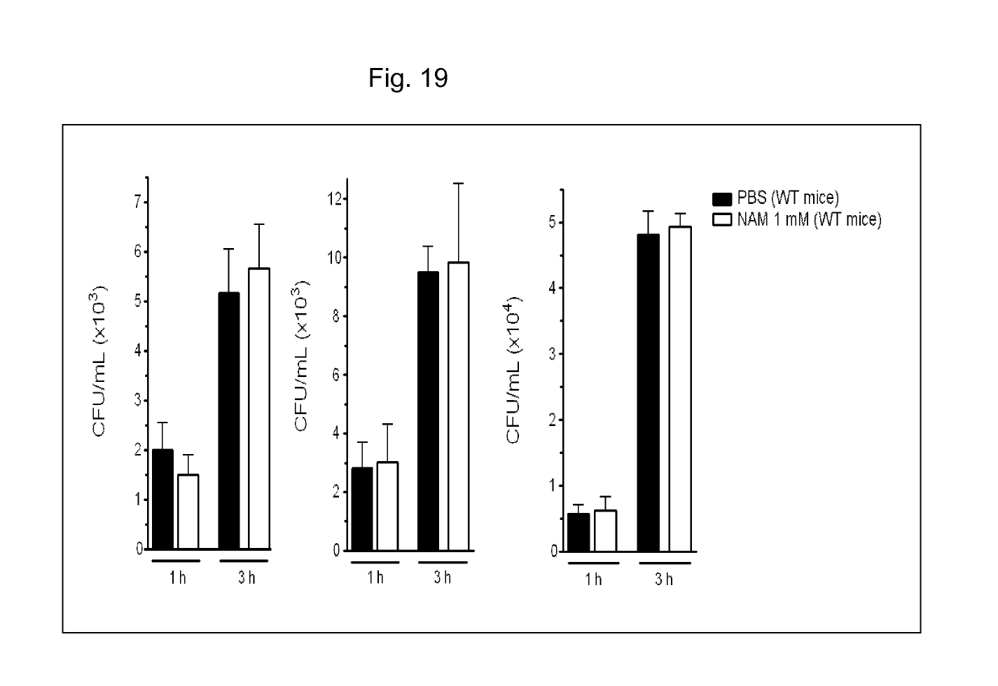

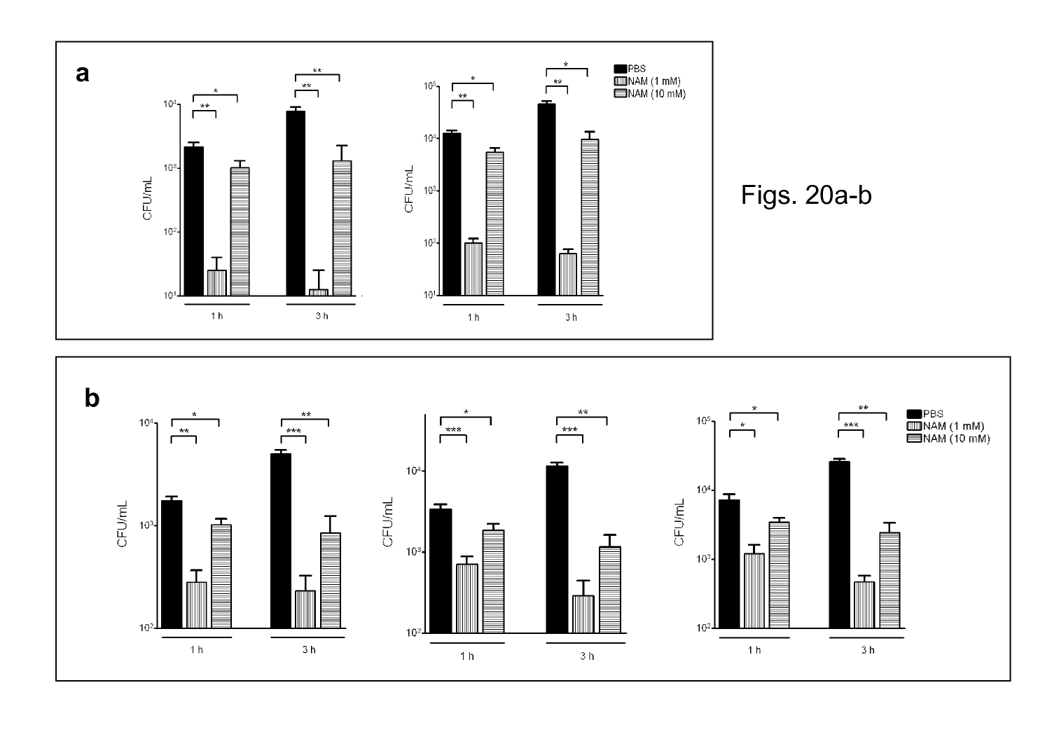

Having demonstrated an important regulatory influence of NAM on the transcriptional activity of C/EBPε in myeloid cells, the inventors asked whether NAM could augment host phagocytic killing of S. aureus. First, the inventors isolated peripheral blood from WT or C/EBPε^{-/-} mice, pretreated the blood for 24 h with either 1 mM NAM or PBS (control), then infected the blood with different inocula of S. aureus. Remarkably in WT groups, NAM pretreatment enhanced killing of S. aureus by more than 3 log₁₀ in compared to PBS controls (FIG. 6a). By contrast in C/EBPε^{-/-} groups, NAM pretreatment had no impact on the number of surviving S. aureus CFU compared to PBS controls (FIG. 6a). Notably, pretreatment of WT murine peripheral blood with NAM for only 4 h (instead of 24 h) did not result in CFU differences (FIG. 19).

To test the in vivo effect of NAM, the inventors injected WT and C/EBPε^{-/-} mice daily with NAM (250 mg/kg i.p.) or PBS starting 24 h prior to systemic (i.p.) infection with S. aureus. This dose has routinely been used in other studies^{38,39}. Strikingly after 48 h, WT mice treated with NAM (compared to PBS) showed approximately 2 logic lower S. aureus CFU in the spleens and kidneys compared to PBS controls (FIG. 6b). Expression levels of CEBPE, LF, and CAMP within isolated BMMC were approximately 2- to 3-fold higher in NAM-treated WT mice compared to PBS-treated mice (FIG. 6c). In contrast to the inventors' findings in WT mice, NAM treatment had no impact on the number of bacterial CFU recovered from the spleens and kidneys of C/EBPε^{-/-} mice at 48 h p.i (FIG. 6d). These findings strongly suggest that C/EBPε and its downstream targets play a role in the immunomodulatory activity of NAM.

To evaluate whether NAM is beneficial for treatment of existing infection, the inventors established systemic infection in WT mice with S. aureus for 12 h prior to commencing daily treatment with NAM (FIG. 6e). After 60 h of infection, the number of bacteria recovered from the spleen and kidneys was 1.5 to 3 log 10 CFU lower in NAM-treated mice compared to PBS-treated controls. These data indicate that NAM can be effective against S. aureus whether the compound is administered before or after infection is established.

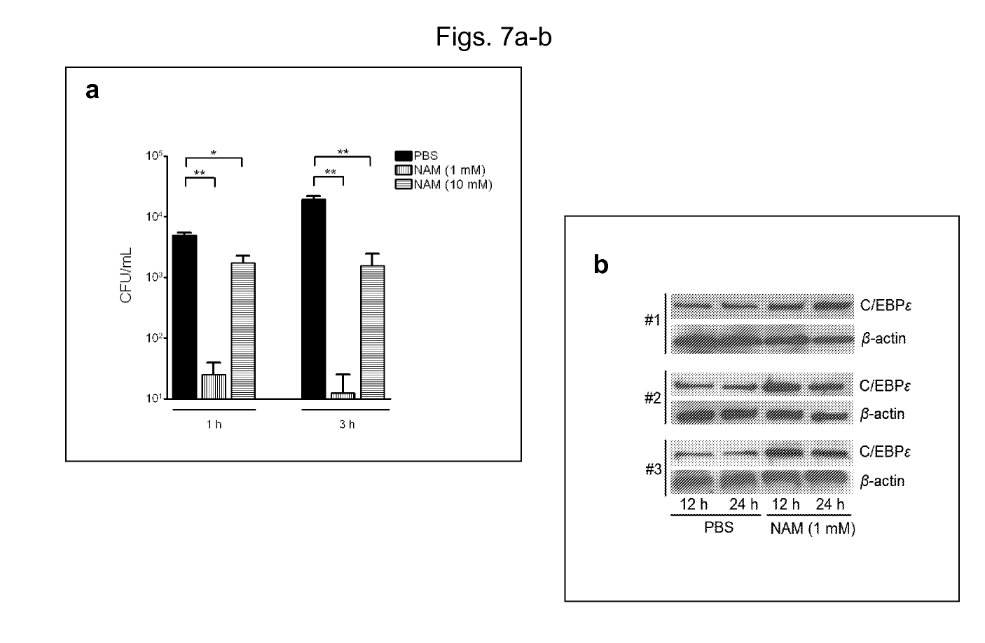

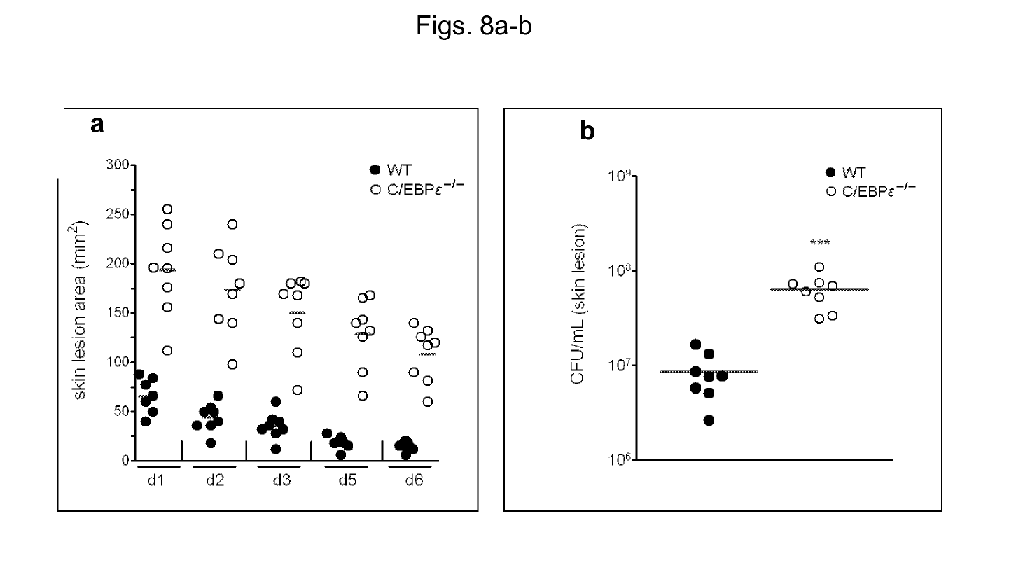

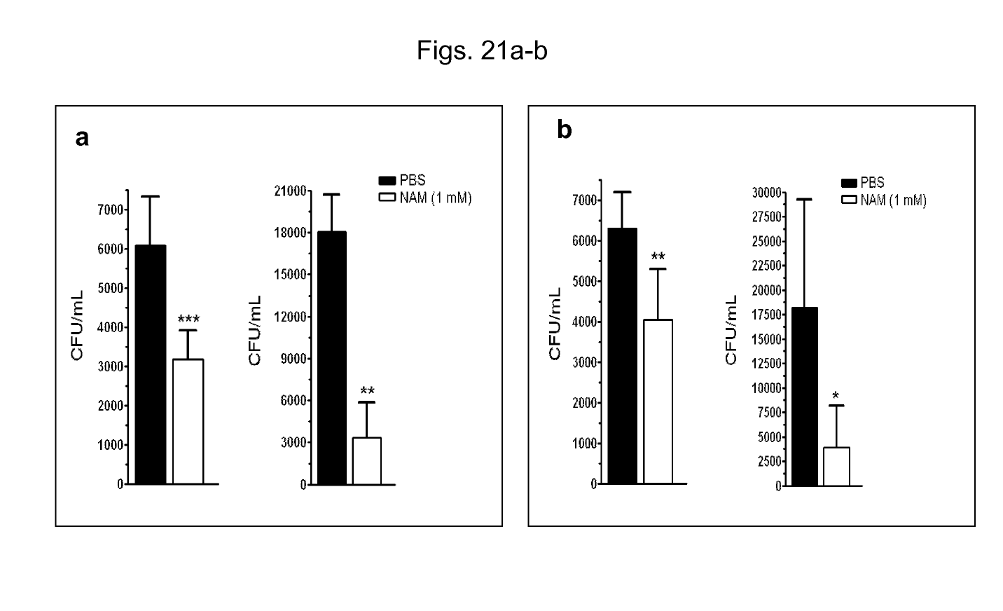

Next, peripheral blood drawn from 12 healthy human volunteers was pretreated ex vivo with NAM (1 mM or 10 mM) or PBS for 24 h prior to infection with different inocula of S. aureus. Consistently, NAM treatment reduced the ability of the pathogen to survive in whole blood by 2-3 log₁₀ at 3 h p.i. compared to PBS treatment (FIG. 7a). Shown in FIG. 7b are the levels of C/EBPε protein extracted from PMNs following NAM treatment of human blood. Consistent with the inventors' findings in mice, 1 mM NAM increased the activity of C/EBPε and improved killing of S. aureus. In line with the inventors' data on S. aureus, NAM pretreatment of human peripheral blood also improved the outcome of infection with other important human pathogens such as K. pneumoniae and P. aeruginosa (FIG. 21).

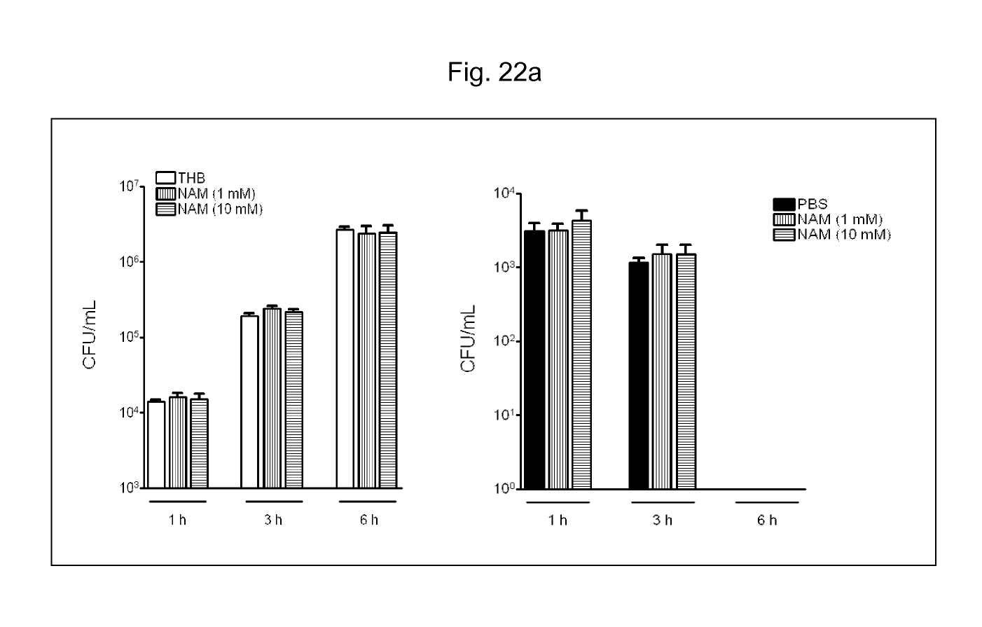

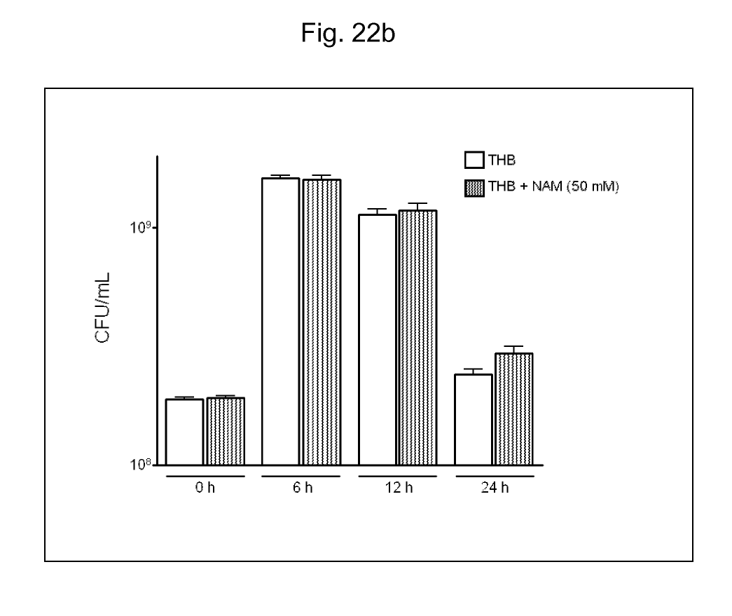

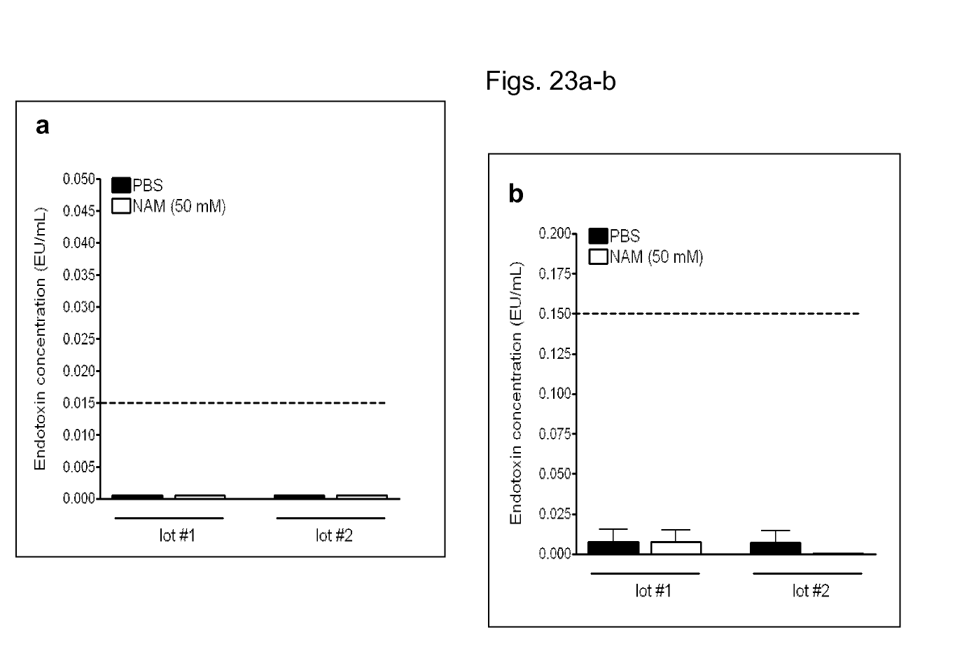

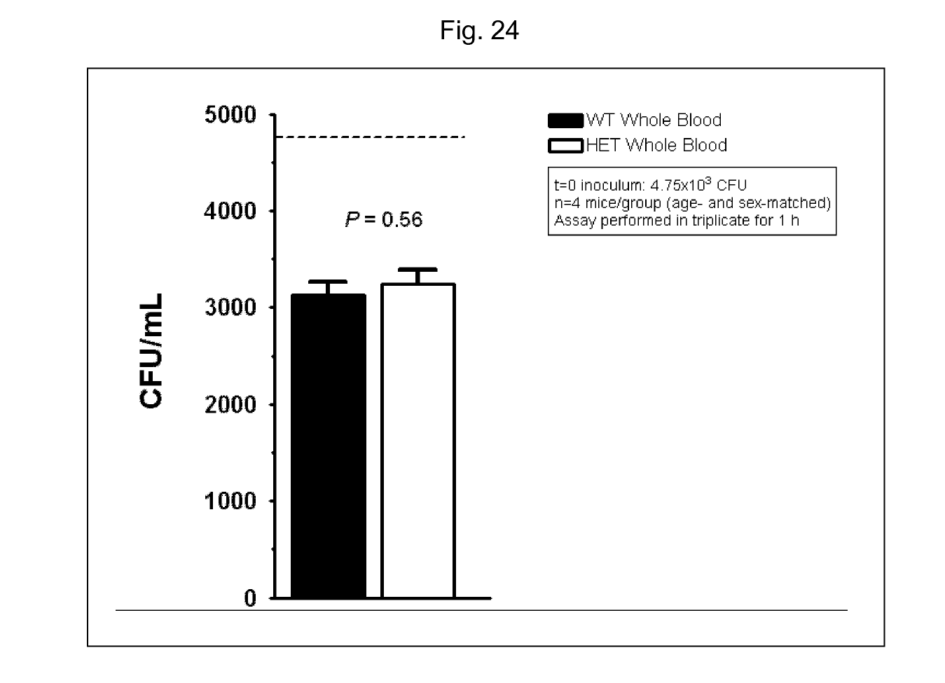

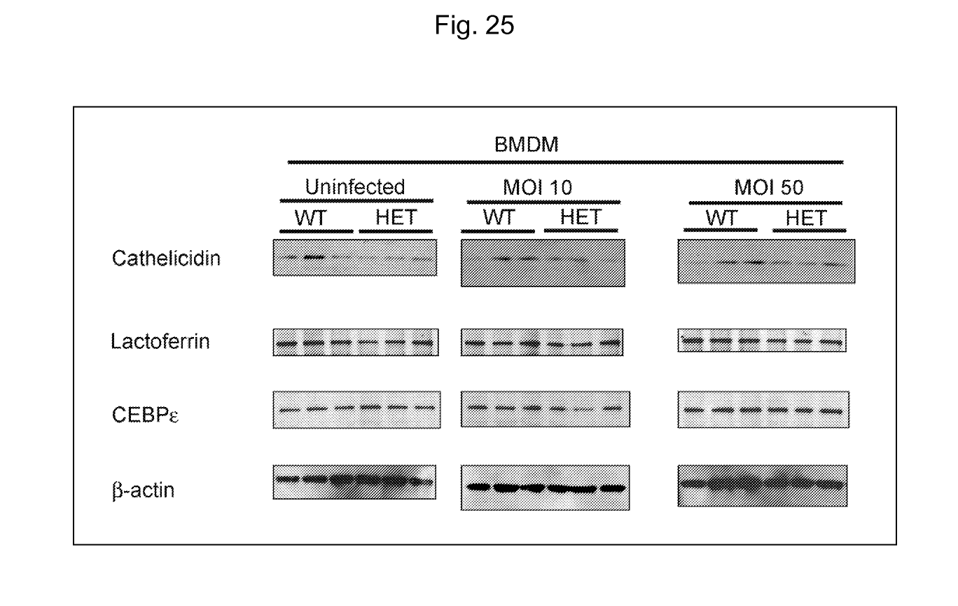

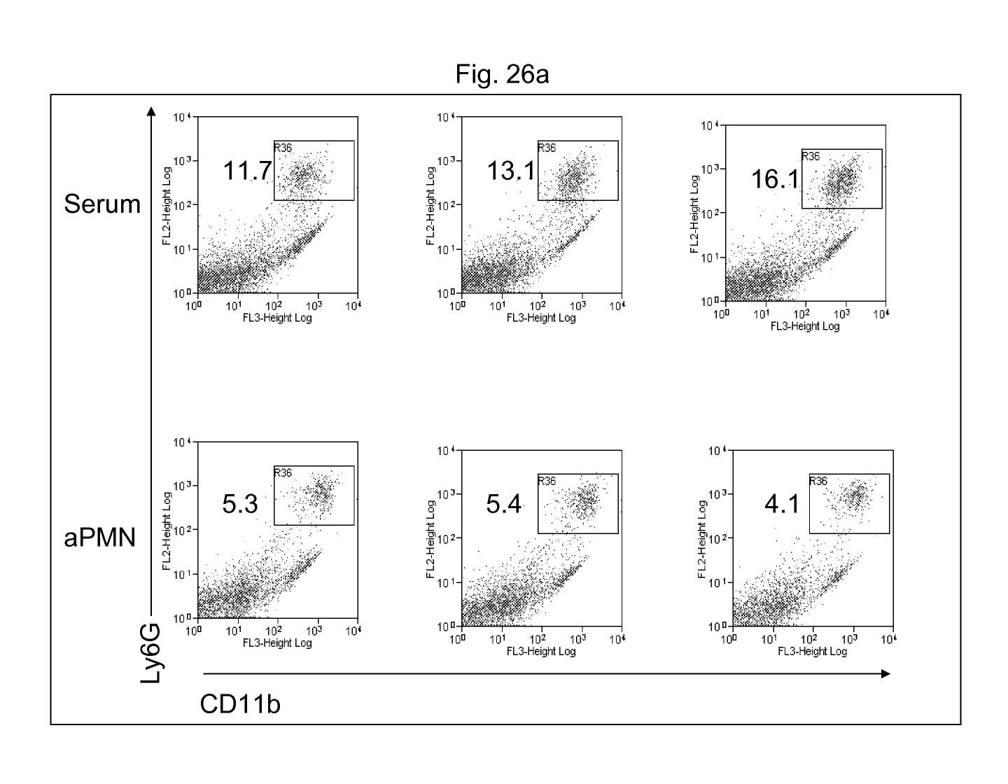

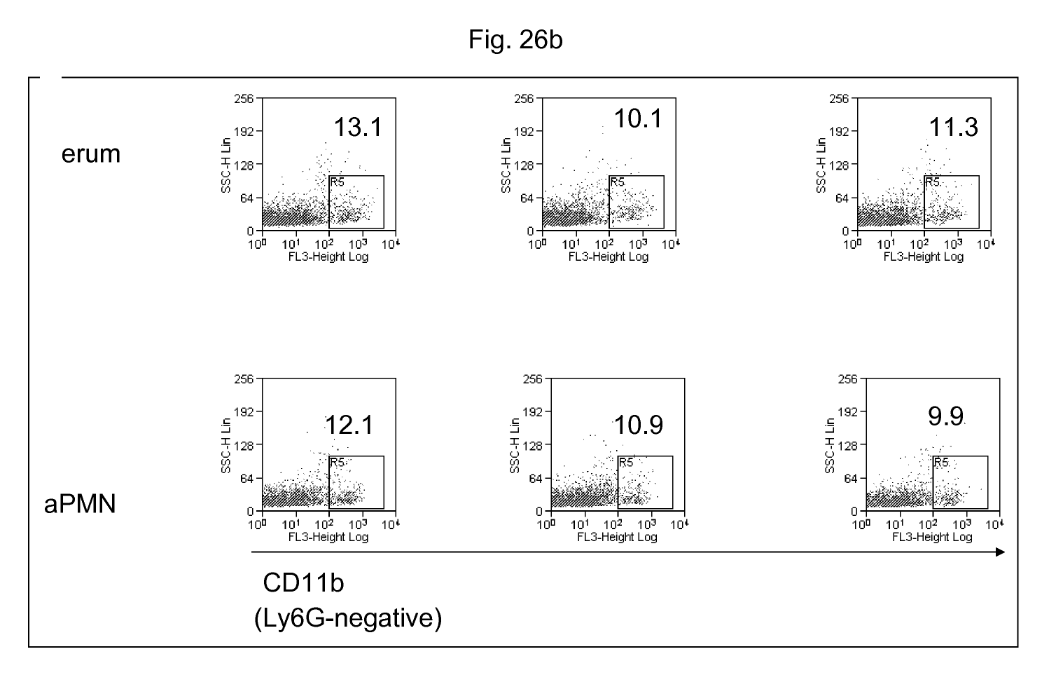

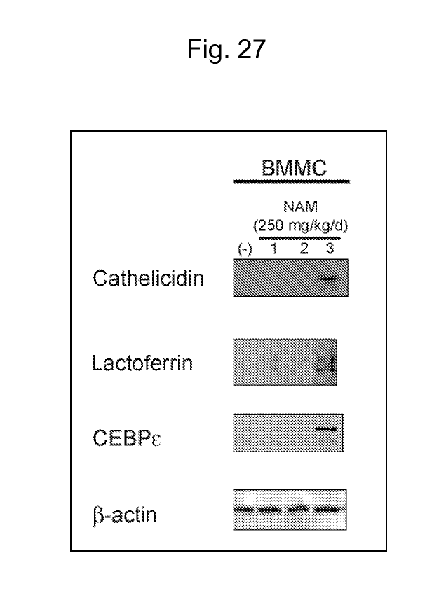

Notably, in the inventors' murine and human ex vivo experiments, the inventors used a NAM concentration that is similar to the plasma concentration previously measured in humans treated with NAM³⁰. NAM importantly had no direct anti-staphylococcal activity when incubated in the absence of phagocytic cells (FIG. 22). As a further control, the NAM used in the inventors' study has been cell culture and insect culture tested, and was confirmed to be endotoxin (pyrogen) free (FIG. 23).

The inventors' findings indicate that increased transcriptional activity of C/EBPε, induced by the epigenetic modulator NAM, can efficiently enhance the clearance of S. aureus both in vitro and in vivo.

### Example 7

### Animals

C/EBPε^{-/-} mice¹² and wild-type (WT) littermates were bred in specific pathogen-free conditions in the animal housing facility at the Burns and Allen Research Institute at Cedars-Sinai Medical Center. Sex-matched mice used throughout the study were 6- to 8-weeks old.

### Example 8

### Bacterial Strains and Growth Conditions

Unless otherwise indicated, WT Staphylococcus aureus Pig1⁴¹, isolated from the skin of a child with atopic dermatitis was used in experiments. The S. aureus clinical isolate LAC (CA-MRSA WT strain USA300; gift from Dr. Binh Diep, UCSF, CA, USA) was also used.

S. aureus were propagated in Todd-Hewitt broth (THB; Difco, Franklin Lakes, N.J., USA) at 37° C. with shaking at 250 rpm or on THB agar (THA). Overnight bacterial culture was diluted 1:500 in prewarmed media and incubated at 37° C. with shaking at 250 rpm until an optical density at 600 nm corresponding to ˜10⁸ CFU/mL was reached. Bacteria were harvested by centrifugation at 3300×g for 10 min at 4° C., and then washed twice with PBS (without Ca²⁺ and Mg²⁺; Mediatech, Manassas, Va. USA). S. aureus strains were routinely cultured on Tryptic Soy sheep blood agar plates and colonies with comparable hemolysis phenotypes were selected for each experiment.

### Example 9

### Murine Skin Infection Model

S. aureus was pelleted, washed twice and resuspended in PBS (Mediatech, Manassas, Va., USA) mixed 1:1 with sterile Cytodex beads (GE Healthcare, Pasadena, Calif., USA) at the specified inoculum, following an established protocol for generating localized S. aureus and S. pyogenes subcutaneous (s.c.) infection^{41,42}. One hundred microliters of two separate inocula, as specified, were administered by s.c. injection into the respective two flanks of each mouse. Injections were performed with careful visualization of the needle to assure that they were not intramuscular. Serial dilutions were prepared and plated to confirm the actual inocula used.

### Example 10

### Determination of Lesion Size and Tissue Bacterial CFU

Baseline weights of mice were recorded prior to infection and daily thereafter until sacrifice. Lesions were measured with a caliper, daily throughout infection. Lesions were defined by darkened areas of dermonecrosis. The inventors' method to measure lesion size has been previously reported⁴³. Briefly, skin lesions were quantified by multiplying the length and width of the lesion. Irregularly-shaped lesions were broken down into smaller symmetrical pieces, and each piece was measured by the same method.

Following euthanization of mice on the specified day, infected skin lesion tissue was aseptically excised, and thoroughly homogenized and mixed in 1 mL of PBS as previously shown⁴³. Ten-fold serial dilutions of the homogenates were plated on THA for CFU determination. The spleen and both kidneys were aseptically removed from each animal and processed in the same way. When required, the appropriate homogenized suspensions (skin lesions) were centrifuged at 15,000 g for 10 min and supernatants stored at −80° C. for subsequent analysis by ELISA.

### Example 11

### Murine Systemic Infection

Mice were systemically infected by intraperitoneal (i.p.) injection of 1×10⁷ CFU/mL S. aureus for 48 h. Following euthanasia, the spleen and both kidneys were removed for CFU determination.

### Example 12

### Nicotinamide (NAM)

Nicotinamide (C₆H₆N₂O; Fw 122.13), tested for cell culture and insect cell culture, was purchased from Sigma (St. Louis, Mo., USA). On each occasion prior to use, NAM was prepared fresh in sterile endotoxin-free PBS (without Ca²⁺ and Mg²⁺), and sterilized through a non-pyrogenic 0.22 μm low-protein binding filter (PALL Life Sciences, Covina, Calif., USA). Each specific lot of NAM used in our study was confirmed to be endotoxin (pyrogen) free using the end-point chromogenic Limulus amebocyte lysate endochrome method.

### Example 13

### Murine and Human Whole Blood Assays

This well described phagocytic survival assay has been previously reported⁴². Bacteria was pelleted, washed twice, diluted to the specified inoculum in 25 μl PBS (without Ca²⁺ and Mg²⁺), and immediately mixed with 75 μl of freshly drawn human or murine peripheral whole blood in sterile heparinized 2 mL round-bottom Eppendorf tubes. When required, murine plasma was obtained by centrifugation of heparinized whole blood at 2000×g for 15 min at RT. Reactions (performed in minimum in triplicate) were incubated at 37° C. for 1-3 h on a rotary shaker, at which time ten-fold serial dilutions were plated on THA for enumeration of surviving CFU.

When required, freshly drawn human or murine peripheral blood was pretreated with NAM (˜0.1 mM or ˜10 mM) or PBS (without Ca²⁺ and Mg²⁺), prior to inoculation with bacteria. Pretreatment was performed in sterile, non-treated, low evaporation tissue culture plates (Becton, Dickinson and Company, Laguna Hills, Calif., USA) for 24 h in a humidified atmosphere at 37° C. and 5% CO₂, with gentle mixing on a nutator.

On each occasion, blood was aseptically taken from mice via cardiac puncture using a 22-gauge needle to minimize lysis and maintain the integrity of the blood for the duration of the respective assay.

### Example 14

### Statistical Analysis

The inventors used two-tailed unpaired student's t-test to compare two independent groups when using ex vivo data; non-parametric Mann-Whitney U test was applied for the independent comparison of the murine in vivo CFU data. One-way ANOVA was used for the comparison of more than two independent groups, and two-way ANOVA, in combination with Bonferroni as post hoc test, to compare murine body weight or lesion size data sets obtained over time. Paired student t-test was employed for the comparison of human blood samples treated either with NAM or PBS. The inventors deemed a P value below 0.05 as significant. GraphPad Prism was used for analyses.

### Example 15

### Quantification of Neutrophils and Macrophages in Infected Skin Lesions

WT and C/EBPε^{-/-} mice were infected by s.c. injection of S. aureus at the specified inoculum, and sacrificed at 24 h p.i. Infected tissues (skin lesions) were then excised and fixed in 10% formalin (Medical Chemical Corporation, Los Angeles, Calif., USA) overnight. Paraffin embedding and hematoxylin and eosin (H&E) staining were performed by the Department of Pathology at Cedars-Sinai Medical Center. Image acquisition was performed with the Zeiss Axio Imager M1 microscope and the AxioVision 4.6 software (Zeiss, Goettingen, Germany). Neutrophils and macrophages were counted separately by two independent observers. The mean (±s.d.) was calculated from ten non-overlapping high power fields within each lesion. A minimum of two mice per genotype were analyzed.

### Example 16

### Enzyme Linked Immunosorbant Assay (ELISA)

Mouse CXCL1 (KC) and CXCL2 (MIP-2) specific ELISAs were performed according to the manufacturer's instructions (R&D Systems, Minneapolis, Minn., USA).

### Example 17

### Neutrophil Depletion In Vivo

Depletion of neutrophils was carried out as described^{44,45}. Briefly, mice were made neutropenic by i.p. administration of 150 μl of rabbit anti-mouse PMN antibody (Cedarlane Laboratories Ltd., Burlington, N.C.) 24 h prior (day −1) to s.c. infection with S. aureus on day 0, and every 24 h thereafter, until sacrifice on day 4. The antibody was certified by the manufacturer to be sterile and suitable for use in cytotoxic assays and in vivo depletion. Control groups received equal amounts of normal rabbit serum (Sigma; sterile-filtered, cell culture and endotoxin tested) by i.p. injection.

WT and C/EBPε^{-/-} mice receiving either anti-mouse PMN antibody or normal serum (control) were infected s.c. with S. aureus on day 0 (Refer to murine skin infection model). Skin lesion areas were measured daily, and on day 4 (sacrifice) the CFU in skin lesions, spleen, and kidneys was determined.

To confirm depletion of neutrophils after antibody injection, WT mice (n=3/group) were sacrificed at day 0 and day 4 of infection. HBSS was injected into the peritoneal cavity, the lavage was collected, and peritoneal exudate cells were stained with Diff-Quick (Siemens Healthcare Diagnostics, Deerfield, Ill., USA). Based on staining and morphology, the total number of neutrophils was determined by microscopy. The total population of neutrophils in the peritoneal exudates of mice treated with antibody versus normal serum was highly reduced on day 0 (−72%, P=0.001) and day 4 (−96%, P=0.008) of infection (data not shown). Additionally, the spleen was collected after sacrifice on day 4, homogenized, and cells stained with PE-anti Ly6G monoclonal antibody and PE.Cy5-anti CD11b monoclonal antibody (eBiosciences, San Diego, Calif., USA). The population of neutrophils (Ly6G⁺, CD11b⁺) were determined by FACScan flow cytometer (BD Biosciences, San Jose, Calif., USA) and the data analyzed by Summit (Dako, Carpinteria, Calif., USA). Again, neutrophils were significantly reduced in the antibody- versus control-treated mice (−64%, P=0.003; data not shown)

### Example 18

### In Vivo Treatment with Interferon Gamma (IFN-γ)

Mice were infected with S. aureus by s.c. injection as already described. One hundred microliters of two separate specified inocula were injected into the respective two flanks of WT and C/EBPε^{-/-} mice.

C/EBPε^{-/-} mice were treated with recombinant murine IFN-γ (Shenandoah Biotechnology, Warwick, Pa., USA) 48 h prior to infection. IFN-γ was administered i.p. (0.5 mL in PBS) at a dose of 5,000 U. Control WT and C/EBPε^{-/-} mice received equal amounts of PBS. The respective mice continued to receive IFN-γ or PBS every 24 h until they were euthanized.

Mouse weight and lesion size were recorded daily as already described. At day 4 p.i., mice were euthanized, and the CFU from skin lesions, spleen, and kidneys were determined.

### Example 19

### Isolation of Murine Bone Marrow Mononuclear Cells (BMMC) and Cultivation of Bone-Marrow Derived Macrophages (BMDM)

Bone marrow cells were harvested from WT or C/EBPε^{-/-} mice. Bone marrow was flushed out of isolated femurs and tibiae with RPMI 1640 medium and 10% heat-inactivated FBS using a 25-gauge needle. Cells were then incubated for 4 h in a humidified atmosphere at 37° C. and 5% CO₂ to deplete adherent cells. BMMC were isolated using Lymphocyte Separation Medium (Mediatech, Manassas, Va., USA) and cultured with 10 ng/mL murine M-CSF (Peprotech, Rocky Hill, N.J., USA) in RPMI 1640 with 10% FBS for 7 days to induce BMDM.

### Example 20

### Development of U937-pMTε Cells

As previously reported by the inventors⁴⁶, the zinc-inducible C/EBPε expression vector (pMTε) was constructed by inserting a full-length of (CEBPE cDNA at the XhoI and HindIII sites of the pMTCB6⁺ vector (pMT; kind gift from F. J. Rauscher, III. The Wistar Institute, Philadelphia, Pa., USA). The inventors used the human pro-monocytic U937 cell line (ATCC, Rockville, Md., USA) stably transfected with pEGFP plasmid (Clontech Laboratories, Palo Alto, Calif., USA) and either zinc-inducible pMTε or control vector pMT⁴³. Cells were maintained between 2×10⁵ and 1×10⁶ cells/mL in RPMI 1640 medium (Invitrogen, Carlsbad, Calif., USA) supplemented with 10% heat-inactivated FBS (Gemini Bio-Products, Sacramento, Calif., USA), 2 mM L-glutamine, and G418 (neomycin, 900 μg/mL; Omega Scientific, Tarzana, Calif., USA) for selection. Multiple polyclonal cultures (>98% GFP positive) were screened for zinc-inducible C/EBPε-overexpression by Western blot analysis.

### Example 21

### Survival Assay in PMA-Differentiated U937 Macrophages

U937 cells alone or carrying either pMTε or vector control, were seeded at a density of 5×10⁴ cells per well (100 μl) in 96-well tissue culture plates. The cells were subsequently induced to differentiate to macrophages by addition of 10 ng/mL of phorbol 12-myristate 13-acetate (PMA; Sigma, St. Louis, Mo., USA) for 24 h. 24 h prior to infection start, media was replaced without G418 and PMA for the remainder of the time. Zinc (100 μM Zn₂SO₄) was added to the respective pMTε and control groups 24 h prior to infection start and was present for the remainder of the assay.

Macrophages were infected with S. aureus at the specified MOI. To promote infection, bacteria were spun down onto the macrophages at 500 g for 10 min at room temperature, before incubating the cells in a humidified atmosphere at 37° C. and 5% CO₂. After 30 min, macrophages were washed three times with pre-warmed media to remove extracellular bacteria. Gentamicin (Invitrogen, Carlsbad, Calif., USA) was then added to each well at a final concentration of 400 μg/mL for 1.5 h. At this time, the concentration of gentamicin in the media was reduced to 100 μg/mL for the remainder of the assay. At 24 h post infection, cells were washed three times with PBS, then 100 μl of 0.02% Triton X-100 in water was added to each well and pipetted vigorously 10× to promote macrophage lysis and release intracellular bacteria. Ten-fold serial dilutions of each cell lysate were immediately plated onto THA, and CFU were enumerated following overnight incubation at 37° C. Data are representative of at least two independent experiments performed in triplicate.

### Example 22

### Survival Assay in BMDM Treated with IFN-7

BMDM harvested from WT and C/EBPε^{-/-} mice were seeded at the required density of 5×10⁴ cells per well (100 μl) in 96-well tissue culture plates. Macrophages were then activated with IFN-γ (200 U/mL) for 48 h prior to infection. Activated and control non-activated macrophages were infected with S. aureus at the specified MOI. The macrophage survival assay was then performed as described for U937 macrophages.

### Example 23

### Real-Time Reverse-Transcriptase Polymerase Chain Reaction (RT-PCR)

For the quantitative mRNA expression analysis of CEBPE, CAMP, and LF, RNA was isolated from murine BMMC or BMDM by the use of the RNeasy Mini Kit (Qiagen, Chatsworth, Calif., USA). Subsequently, cDNAs were synthesized from high quality RNA samples with an oligo(dT) primer and random hexamers using Superscript III reverse transcriptase according to the manufacturer's recommendation (Invitrogen, Carlsbad, Calif., USA). Gene expression was quantified with real-time RT-PCR (iCycler, Bio-Rad, Hercules, Calif., USA) using HotMaster Taq DNA Polymerase (Eppendorf, Hamburg, Germany) and SYBRGreen I (Molecular Probes, Eugene, Oreg., USA). Reactions were performed in triplicates using an iCycler iQ system (Bio-Rad). Sequences of the primer sets were used as followed: CEBPE: 5′-GGG CAA CCG AGG CAC CAG TC-3′ (forward) (SEQ ID NO: 1), 5′-CGC CTC TTG GCC TTG TCC CG-3′ (reverse) (SEQ ID NO: 2); LF: 5′-GAG CTG TGT TCC CGG TGC CC-3′ (forward) (SEQ ID NO: 3), 5′-CCG TGC TTC CTC TGG TAA AAG CCA-3′ (reverse) (SEQ ID NO: 4); CAMP: 5′-ACT CCC AAG TCT GTG AGG TTC CGA-3′ (forward) (SEQ ID NO: 5), 5′-TGT CAA AAG AAT CAG CGG CCG GG-3′ (reverse) (SEQ ID NO: 6); 1)-actin: 5′-GGA CTT CGA GCA AGA GAT GG-3′ (forward) (SEQ ID NO: 7). 5′-CCG CCA GAC AGC ACT GTG TT-3′ (reverse) (SEQ ID NO: 8). For each sample, the amount of the target genes and reference gene was determined using standard curves. mRNA levels were normalized against endogenous β-actin. The results of real-time RT-PCR are presented as mean±s.d. using either BMDM obtained from 3 mice or BMMC from 4 mice per experiment.

### Example 24

### Immunnnoprecipitation (IP) and Western Blotting

Whole-cell extracts were produced by lysing cells (10⁷) with 100 mL denaturing RIPA buffer (50 mM Tris HCl pH 8, 150 mM NaCl, 1%, NP-40, 0.5% sodium deoxycholate, 0.1% SDS) added with a protease inhibitor cocktail (Roche Molecular Biochemicals, Indianapolis, Ind., USA) on the day of extraction. Extracts were stored at −80° C. until use.

The inventors used an anti-acetyl-lysine antibody (ab21623; Abcam, Cambridge, Mass., USA) for IP according to the manufacturer's protocol. The input of the protein lysates was used as a loading control.

For Western blot, protein lysates were boiled in Laemmli sample buffer (Bio-Rad), resolved on 4% to 15% gradient sodium dodecyl sulfate-polyacrylamide (SDS-RAGE) gels and transferred to nitrocellulose membranes (Sigma, St. Louis, Mo., USA). Immunoblots were probed with C/EBPε-antibody (Santa Cruz Biotechnology, Santa Cruz, Calif., USA) and developed using the enhanced chemiluminescence kit (Pierce, Rockford, Ill., USA). β-actin (Sigma) was used as a control. Western blot data are representative of one out of three independently performed experiments. Densitometry of all blots was performed using the Quantity One software 4.6.3 (Bio-Rad).

### Example 25

### Chromatin Immunoprecipitation (ChIP)

ChIP assay kit (Upstate Biotechnology, Lake Placid, N.Y.) was used, and chromatin was prepared for IP as instructed by the manufacturer. The sonicated chromatin was immunoprecipitated with either 5 μg of anti-acetylated histone H3 antibody or normal rabbit IgG antibody as negative control (Upstate Biotechnology). Immunoprecipitated DNA was subsequently analyzed by PCR using primers specific for the CEBPE promoter region; input chromatin was analyzed for β-actin mRNA as a positive control. The optimal reaction conditions for PCR were determined for each primer pair. Primers were denatured at 95° C. for 1 min and annealed at 60° C. for 1 min, followed by elongation at 72° C. for 1 min; each product was amplified 35 cycles. PCR products were analyzed by 2.5% agarose/ethidium bromide gel electrophoresis. Densitometry of all agarose gels was performed using the Quantity One software 4.6.3 (Bio-Rad). The primers used for ChIP analysis were: human CEBPE 5′-GCT TTG GCC AAG CCC AGG GA-3′ (forward) (SEQ ID NO: 9), 5′-TGC TGG GCT CCA CCT ACC CC-3′ (reverse) (SEQ ID NO: 10); human β-actin: 5′-CTC CTC GGG AGC CAC ACG CA-3′ (forward) (SEQ ID NO: 1), 5′-TAG GGG AGC TGG CTG GGT GG-3′ (reverse) (SEQ ID NO: 12); murine CEBPE: 5′-TGA GGC TGC AGC TTG CCT GG-3′ (forward) (SEQ ID NO: 13), 5′-ACC AAG CTA CCC CTG GCC CT-3′ (reverse) (SEQ ID NO: 14), murine β-actin: 5′-ACC TGT TAC TTT GGG AGT GGC AAG C-3′ (forward) (SEQ ID NO: 15), 5′-GTC GTC CCA GTT GCT AAC AAT GCC-3′ (reverse) (SEQ ID NO: 16).

### Example 26

### Transient Transfection and Luciferase Assays

The inventors designed a −230 LF promoter reporter plasmid (LAC-LUC) including a C/EBP-binding site as previously described¹⁵. For the reporter gene assay, 2×10⁶ U1937 cells were transiently transfected either with 2 μg of the LAC-LUC luciferase reporter gene constructs or the empty-vector control (pGL3, Promega, Madison, Wis., USA), as well as 0.2 μg of Renilla luciferase (pRL-SV40). Transfections were performed using the nucleofection technique with the Amaxa-Kit (Invitrogen, Karlsruhe, Germany) according to the manufacturer's instructions. After 16 h of transfection, the cells were treated with NAM (1 mM) for an additional 16 h. The lysates were harvested and luciferase activity measured by the Dual-Luciferase reporter assay system (Promega, Madison, Wis., USA). For all transfection studies, luciferase activity was normalized using pRL-SV40 activity. Results represent the mean of three independent experiments performed in triplicate.

### Example 27

### Isolation of Human Blood and PMNs

Participants in the inventors' study included 15 healthy humans with a negative history of infection or antibiotic treatments in the prior 4 weeks. Peripheral blood was collected from individuals in a fasting condition. Polymorph-prep (Axis-Shield, Oslo, Norway) was used for the isolation of PMNs according to the manufacturer's protocol.

### Example 28

### Assessing the Effect of NAM and Zinc on the Growth and Viability of S. Aureus

The inventors assessed whether NAM, at the concentrations used in the study, adversely affect the growth and viability of S. aureus. PBS was chosen as an inert non-growth medium, and THB was chosen as a suitable growth medium for S. aureus. Seventy-five microliters of NAM (1 mM and 10 mM final concentrations; in either THB or PBS) or THB or PBS alone (respective controls) were placed in sterile 2 mL round-bottom Eppendorf tubes, and then inoculated with S. aureus (˜1×10⁴ CFU/mL in 25 μl of PBS or THB) and immediately briefly vortexed. Triplicate reactions were incubated at 37° C. for 1 h, 3 h, and 6 h on a rotary shaker at which time ten-fold serial dilutions were plated on THIA for enumeration of CFU.

In a different assay, S. aureus (˜1×10⁸ CFU/mL in THB) was incubated with or without 50 mM NAM. This concentration represents the equivalent molarity of NAM used in the in vivo experiments. Triplicate 1 mL reactions were incubated at 37° C. on a rotary shaker and at various time points (6 h, 12 h, and 24 h) ten-fold serial dilutions were plated on THA for enumeration of CFU. This assay was performed on two independent occasions using inocula generated from three separate bacterial cultures.

To assess whether zinc, at the concentrations used in the inventors' study, adversely affect the growth and viability of S. aureus (strains Pig1 and LAC), bacteria (˜5×10⁵ CFU) were incubated with or without 100 μM zinc (Zn₂ SO₄) in RPMI 1640 with 10% FBS. The assay was performed in triplicate in 96-well plates for 24 h at 37° C. at which time ten-fold serial dilutions were plated on THA for enumeration of CFU.

### Example 29

Verification that the NAM Used in the Study is Free of Detectable Endotoxin

To confirm the absence of endotoxin (pyrogen), the inventors tested the two lots of NAM used in the study. The quantitative detection of bacterial endotoxin in aqueous solutions of NAM was determined by end-point chromogenic Limulus amebocyte lysate endochrome method (Endosafe; Charles River, San Diego, Calif., USA). Non-LAL reactive LAL reagent water was used as diluent for preparing reagents and test specimen. Two separate microplate assays were performed measuring high concentration range (0.15-1.2 EU/mL) and low concentration range (0.015-0.12 EU/mL). The linearity of the standard curve within the concentration range used to determine endotoxin levels was verified. At least 4 endotoxin standards, spanning the desired concentration range, and an endotoxin-free water blank were assayed in quadruplicate. The absolute value of the coefficient of correlation, r, was greater than or equal to 0.980. Replicate samples were run to establish proficiency and low coefficient of variation. The coefficient of variation, CV, which is equal to 100 times the s.d. of the group of values, divided by the mean, was less than the allowed 10%.

### Example 30

### Genotyping of Mice

First, mouse tail tips were digested in buffer containing 10 mM Tris-HCl (pH 8.0), 100 mM EDTA, 0.5% SDS and 0.1 mg/mL proteinase K (Sigma-Aldrich, St. Louis, Mo., USA), overnight at 50° C. Genomic DNA was then isolated by phenol/chloroform extraction followed by ethanol precipitation, and resuspended in 1 mL of Tris/EDTA buffer (pH 8). To determine the genotype of mice, 3 primers termed Neo1500 5′-ATC GCC TTC TAT CGC CTT CTT GAC GAG-3′ (SEQ ID NO: 17), mepsilon S 5′-GCT ACA ATC CCC TGC AGT ACC-3′ (SEQ ID NO: 18) and mepsilon AS 5′-TGC CTT CTT GCC CTT GTG-3′ (SEQ ID NO: 19) were utilized. To detect each allele, the following combinations of primers were used: mepsilon S and mepsilon AS for the WT allele, and mepsilon S and Neo1500 for the knockout allele of the CEBPE gene. Genomic PCR was performed using the FailSafe PCR buffer PreMix F (Epicentre Biotechnologies, Madison, Wis., USA).

### Example 31

### Additional Pathogens

TABLE 1
Category A
Bacillus anthracis (anthrax)
Clostridium botulinum toxin (botulism)
Yersinia pestis (plague)
Variola major (smallpox) and other related pox viruses
Francisella tularensis (tularemia)
Viral hemorrhagic fevers
Arenaviruses

LCM, Junin virus, Machupo virus, Guanarito virus
Lassa Fever

Bunyaviruses

Hantaviruses
Rift Valley Fever

Flaviruses

Dengue

Filoviruses

Ebola
Marburg

Category B

Burkholderia pseudomallei
Coxiella burnetii (Q fever)
Brucella species (brucellosis)
Burkholderia mallei (glanders)
Chlamydia psittaci (Psittacosis)
Typhus fever (Rickettsia prowazekii)
Food- and Waterborne Pathogens

Bacteria

Diarrheagenic E. coli
Pathogenic Vibrios
Shigella species
Salmonella
Listeria monocytogenes
Campylobacter jejuni
Yersinia enterocolitica)

Viruses (Caliciviruses, Hepatitis A)
Protozoa

Cryptosporidium parvum
Cyclospora cayatanensis
Giardia lamblia
Entamoeba histolytica
Toxoplasma

Fungi

Microsporidia

Additional viral encephalitides

West Nile Virus
LaCrosse
California encephalitis
VEE
EEE
WEE
Japanese Encephalitis Virus
Kyasanur Forest Virus

Category C
Emerging infectious disease threats such as Nipah virus and
additional hantaviruses.
NIAID priority areas:
Tickborne hemorrhagic fever viruses

Crimean-Congo Hemorrhagic fever virus

Tickborne encephalitis viruses
Yellow fever
Tuberculosis, including drug-resistant TB
Influenza
Other Rickettsias
Rabies
Prions
Chikungunya virus
Severe acute respiratory syndrome associated coronavirus (SARS-CoV)
Coccidioides immitis (added February 2008)
Coccidioides posadasii (added February 2008)
*NIAID Category C Antimicrobial Resistance-Sexually Transmitted
Excluded Organisms
Bacterial vaginosis, Chlamydia trachomatis, Cytomegalovirus,
Granuloma inguinale, Hemophilus ducreyi, Hepatitis B virus,
Hepatitis C virus, Herpes Simplex virus, Human immunodeficiency virus,
Human papillomavirus, Neisseria gonorrhea, Treponema pallidum,
Trichomonas vaginalis

### Example 32

### Peripheral Whole Blood Studies

WT mice treated in vivo with NAM (250 mg/kg/day; i.p.) or PBS (control) for a) 24 h, b) 48 h, and c) 72 h. Then, peripheral whole blood was removed from each of the mice and CBCs were performed. No differences in CBCs were observed at any timepoint, between NAM- and PBS-treated mice. (Table 2A-C)

TABLE 2A

| | | Leukocytes: | | | | |
|---|---|---|---|---|---|---|
| Parameter (Units) | Normal Range | 24p1 | 24p2 | 24p3 | 24p4 | 24p5 |
| WBC (K/uL) | 1.8-10.7 | 8.66 | 4.60 | 4.34 | 4.68 | 3.50 |
| NE (K/uL) | 0.1-2.4 | 1.70 | 1.03 | 1.15 | 1.17 | 0.73 |
| LY (K/uL) | 0.9-9.3 | 6.34 | 3.19 | 2.87 | 3.17 | 2.58 |
| MO (K/uL) | 0.0-0.4 | 0.50 | 0.25 | 0.22 | 0.19 | 0.16 |
| NE (%) | 6.6-38.9 | 19.65 | 22.49 | 26.56 | 25.01 | 20.73 |
| LY (%) | 55.8-91.6 | 73.20 | 69.36 | 66.15 | 67.78 | 73.81 |
| MO (%) | 0.0-7.5 | 5.79 | 5.44 | 5.18 | 4.11 | 4.45 |

| | | | | | |
|---|---|---|---|---|---|
| Hematological Abnormalities | Monocytosis | Normal | Normal | Normal | Normal |

| | | Leukocytes: | | | | |
|---|---|---|---|---|---|---|
| Parameter (Units) | Normal Range | 24p6 | 24p7 | 24p8 | 24p9 | 24p10 |
| WBC (K/uL) | 1.8-10.7 | 7.42 | 4.50 | 2.46 | 3.22 | 2.30 |
| NE (K/uL) | 0.1-2.4 | 2.07 | 0.47 | 0.65 | 0.98 | 0.79 |
| LY (K/uL) | 0.9-9.3 | 4.62 | 3.72 | 1.59 | 2.00 | 1.21 |
| MO (K/uL) | 0.0-0.4 | 0.38 | 0.25 | 0.16 | 0.21 | 0.23 |
| NE (%) | 6.6-38.9 | 27.85 | 10.35 | 26.49 | 30.51 | 34.35 |
| LY (%) | 55.8-91.6 | 62.30 | 82.71 | 64.57 | 62.07 | 52.61 |
| MO (%) | 0.0-7.5 | 5.06 | 5.51 | 6.70 | 6.47 | 10.00 |

| | | | | | |
|---|---|---|---|---|---|
| Hematological Abnormalities | Normal | Normal | Normal | Normal | Normal |

| | | Leukocytes: | | | |
|---|---|---|---|---|---|
| Parameter (Units) | Normal Range | 24p11 | 24p12 | Mean | S.D. |
| WBC (K/uL) | 1.8-10.7 | 2.26 | 2.08 | 4.17 | 2.069 |
| NE (K/uL) | 0.1-2.4 | 0.76 | 0.67 | 1.01 | 0.454 |
| LY (K/uL) | 0.9-9.3 | 1.28 | 1.25 | 2.82 | 1.548 |
| MO (K/uL) | 0.0-0.4 | 0.11 | 0.12 | 0.23 | 0.111 |
| NE (%) | 6.6-38.9 | 33.57 | 32.17 | 25.81 | 6.844 |
| LY (%) | 55.8-91.6 | 56.81 | 59.91 | 65.94 | 8.194 |
| MO (%) | 0.0-7.5 | 4.68 | 5.90 | 5.77 | 1.537 |

| | | |
|---|---|---|
| Hematological Abnormalities | Normal | Normal |

| | | Leukocytes: | | | | |
|---|---|---|---|---|---|---|
| Parameter (Units) | Normal Range | 24n1 | 24n2 | 24n3 | 24n4 | 24n5 |
| WBC (K/uL) | 1.8-10.7 | 3.30 | 2.18 | 2.12 | 2.94 | 3.80 |
| NE (K/uL) | 0.1-2.4 | 0.54 | 0.31 | 0.61 | 1.20 | 1.65 |
| LY (K/uL) | 0.9-9.3 | 2.49 | 1.72 | 1.38 | 1.50 | 1.96 |
| MO (K/uL) | 0.0-0.4 | 0.25 | 0.12 | 0.09 | 0.19 | 0.12 |
| NE (%) | 6.6-38.9 | 16.22 | 14.42 | 28.88 | 40.66 | 43.33 |
| LY (%) | 55.8-91.6 | 75.37 | 78.97 | 65.33 | 51.05 | 51.65 |
| MO (%) | 0.0-7.5 | 7.52 | 5.55 | 4.22 | 6.35 | 3.11 |

| | | | | | |
|---|---|---|---|---|---|
| Hematological Abnormalities | Normal | Normal | Normal | Normal | Normal |

| | | Leukocytes: | | |
|---|---|---|---|---|
| Parameter (Units) | Normal Range | 24n6 | 24n7 | 24n8 |
| WBC (K/uL) | 1.8-10.7 | 5.16 | 2.78 | 5.38 |
| NE (K/uL) | 0.1-2.4 | 0.95 | 0.65 | 1.00 |
| LY (K/uL) | 0.9-9.3 | 3.97 | 1.89 | 3.84 |
| MO (K/uL) | 0.0-0.4 | 0.19 | 0.18 | 0.28 |
| NE (%) | 6.6-38.9 | 18.47 | 23.40 | 18.67 |
| LY (%) | 55.8-91.6 | 76.92 | 67.88 | 71.36 |
| MO (%) | 0.0-7.5 | 3.77 | 6.47 | 5.28 |

| | | | |
|---|---|---|---|
| Hematological Abnormalities | Normal | Normal | Normal |

| | | Leukocytes: | | | | |
|---|---|---|---|---|---|---|
| Parameter (Units) | Normal Range | 24n9 | 24n10 | Mean | S.D. | P value |
| WBC (K/uL) | 1.8-10.7 | 4.24 | 4.44 | 3.63 | 1.162 | 0.4561 |
| NE (K/uL) | 0.1-2.4 | 0.90 | 0.83 | 0.86 | 0.377 | 0.4126 |
| LY (K/uL) | 0.9-9.3 | 3.13 | 3.11 | 2.50 | 0.958 | 0.5613 |
| MO (K/uL) | 0.0-0.4 | 0.17 | 0.36 | 0.20 | 0.082 | 0.3836 |
| NE (%) | 6.6-38.9 | 21.34 | 18.63 | 24.40 | 10.116 | 0.7130 |
| LY (%) | 55.8-91.6 | 73.82 | 70.08 | 68.24 | 9.803 | 0.5623 |
| MO (%) | 0.0-7.5 | 4.03 | 8.11 | 5.44 | 1.669 | 0.6346 |

| | | |
|---|---|---|
| Hematological Abnormalities | Normal | Normal |

Key (Sample ID and results)
24—24 h post-treatment
n—NAM (250 mg/kg/d)
p—PBS treatment (control)
1, 2, 3, 4 . . . mouse number
HEMAVET 950FS, DREW Scientific Inc, Oxford, CT
MASCOT HEMATOLOGY PROFILE
Species: mouse (129/Sv-E, wildtype)
Date of test: Mar. 15, 2011
Date of test: Apr. 05, 2011

TABLE 2B

| | | Results | | | | | | |
|---|---|---|---|---|---|---|---|---|
| | | Leukocytes: | | | | | | |
| Parameter (Units) | Normal Range | 48p1 | 48p2 | 48p3 | 48p4 | 48p5 | mean | S.D. |
| WBC (K/uL) | 1.8-10.7 | 3.46 | 1.88 | 1.86 | 2.30 | 2.22 | 2.34 | 0.654 |
| NE (K/uL) | 0.1-2.4 | 0.60 | 0.60 | 0.40 | 0.53 | 0.41 | 0.51 | 0.098 |
| LY (K/uL) | 0.9-9.3 | 2.55 | 1.13 | 1.26 | 1.51 | 1.57 | 1.60 | 0.559 |
| MO (K/uL) | 0.0-0.4 | 0.23 | 0.12 | 0.18 | 0.21 | 0.17 | 0.18 | 0.042 |
| NE (%) | 6.6-38.9 | 17.29 | 32.16 | 21.51 | 23.11 | 18.40 | 22.49 | 5.887 |
| LY (%) | 55.8-91.6 | 73.59 | 60.24 | 67.59 | 65.57 | 70.92 | 67.58 | 5.128 |
| MO (%) | 0.0-7.5 | 6.52 | 6.62 | 9.75 | 8.97 | 7.47 | 7.87 | 1.440 |

| | | | | | |
|---|---|---|---|---|---|
| Hematological Abnormalities | Normal | Normal | Normal | Normal | Normal |

| | | Results | | | | | | | |
|---|---|---|---|---|---|---|---|---|---|
| | | Leukocytes: | | | | | | | |
| Parameter (Units) | Normal Range | 48n1 | 48n2 | 48n3 | 48n4 | 48n5 | mean | S.D. | P-value |
| WBC (K/uL) | 1.8-10.7 | 6.34 | 1.86 | 3.30 | 4.64 | 2.70 | 3.77 | 1.759 | 0.1495 |
| NE (K/uL) | 0.1-2.4 | 1.41 | 0.29 | 0.59 | 1.01 | 0.58 | 0.78 | 0.438 | 0.2464 |
| LY (K/uL) | 0.9-9.3 | 4.37 | 1.41 | 2.41 | 3.16 | 1.81 | 2.63 | 1.174 | 0.1300 |
| MO (K/uL) | 0.0-0.4 | 0.34 | 0.14 | 0.12 | 0.26 | 0.20 | 0.21 | 0.090 | 0.5265 |
| NE (%) | 6.6-38.9 | 22.22 | 15.43 | 17.76 | 21.75 | 21.48 | 19.73 | 2.988 | 0.3854 |
| LY (%) | 55.8-91.6 | 68.85 | 75.58 | 73.06 | 68.03 | 57.04 | 70.51 | 3.645 | 0.3313 |
| MO (%) | 0.0-7.5 | 5.37 | 7.27 | 3.72 | 5.50 | 7.40 | 5.85 | 1.526 | 0.0642 |

| | | | | | |
|---|---|---|---|---|---|
| Hematological Abnormalities | Normal | Normal | Normal | Normal | Normal |

Key (Sample ID and results)
48—48 h post-treatment
n—NAM (250 mg/kg/d)
p—PBS treatment (control)
1, 2, 3, 4 . . . mouse number
HEMAVET 950FS, DREW Scientific Inc, Oxford, CT
MASCOT HEMATOLOGY PROFILE
Species: mouse (129/Sv-E, wildtype)
Date of test: Mar. 16, 2011

TABLE 2C

| | | Leukocytes: | | | | |
|---|---|---|---|---|---|---|
| Parameter (Units) | Normal Range | 72p1 | 72p2 | 72p3 | 72p4 | 72p5 |
| WBC (K/uL) | 1.8-10.7 | 4.40 | 7.18 | 6.60 | 3.02 | 6.28 |
| NE (K/uL) | 0.1-2.4 | 0.47 | 1.43 | 1.68 | 1.16 | 2.47 |
| LY (K/uL) | 0.9-9.3 | 3.52 | 5.29 | 4.56 | 1.71 | 3.53 |
| MO (K/uL) | 0.0-0.4 | 0.39 | 0.25 | 0.31 | 0.10 | 0.17 |
| NE (%) | 6.6-38.9 | 10.63 | 19.93 | 25.49 | 38.30 | 39.28 |
| LY (%) | 55.8-91.6 | 79.96 | 73.64 | 69.13 | 56.75 | 56.22 |
| MO (%) | 0.0-7.5 | 8.85 | 3.53 | 4.77 | 3.26 | 2.71 |

| | | | | | |
|---|---|---|---|---|---|
| Hematological Abnormalities | Normal | Normal | Normal | Normal | NEUTROPHILIA |

| | | Leukocytes: | | | | |
|---|---|---|---|---|---|---|
| Parameter (Units) | Normal Range | 72p6 | 72p7 | 72p8 | Mean | S.D. |
| WBC (K/uL) | 1.8-10.7 | 5.32 | 6.34 | 5.80 | 5.62 | 1.348 |
| NE (K/uL) | 0.1-2.4 | 0.99 | 1.39 | 2.31 | 1.49 | 0.664 |
| LY (K/uL) | 0.9-9.3 | 3.92 | 4.47 | 3.19 | 3.77 | 1.079 |
| MO (K/uL) | 0.0-0.4 | 0.36 | 0.35 | 0.24 | 0.27 | 0.100 |
| NE (%) | 6.6-38.9 | 18.64 | 21.98 | 39.89 | 26.77 | 11.081 |
| LY (%) | 55.8-91.6 | 73.72 | 70.48 | 54.93 | 66.85 | 9.567 |
| MO (%) | 0.0-7.5 | 6.70 | 5.49 | 4.15 | 4.93 | 2.040 |

| | | | |
|---|---|---|---|
| Hematological Abnormalities | Normal | Normal | Normal |

| | | Leukocytes: | | | | |
|---|---|---|---|---|---|---|
| Parameter (Units) | Normal Range | 72n1 | 72n2 | 72n3 | 72n4 | 72n5 |
| WBC (K/uL) | 1.8-10.7 | 2.20 | 4.08 | 5.66 | 2.24 | 3.62 |
| NE (K/uL) | 0.1-2.4 | 0.51 | 0.86 | 1.05 | 0.62 | 1.22 |
| LY (K/uL) | 0.9-9.3 | 1.60 | 2.88 | 4.34 | 1.35 | 2.10 |
| MO (K/uL) | 0.0-0.4 | 0.06 | 0.24 | 0.26 | 0.22 | 0.22 |
| NE (%) | 6.6-38.9 | 23.25 | 20.96 | 18.47 | 27.89 | 33.59 |
| LY (%) | 55.8-91.6 | 72.55 | 70.58 | 76.68 | 60.42 | 57.97 |
| MO (%) | 0.0-7.5 | 2.89 | 5.82 | 4.53 | 9.61 | 6.08 |

| | | | | | |
|---|---|---|---|---|---|
| Hematological Abnormalities | Normal | Normal | Normal | Normal | Normal |

| | | Leukocytes: | | | | | |
|---|---|---|---|---|---|---|---|
| Parameter (Units) | Normal Range | 72n6 | 72n7 | 72n8 | Mean | S.D. | P-value |
| WBC (K/uL) | 1.8-10.7 | 6.22 | 3.06 | 5.48 | 4.07 | 1.568 | 0.0531 |
| NE (K/uL) | 0.1-2.4 | 2.28 | 0.97 | 1.84 | 1.17 | 0.606 | 0.3329 |
| LY (K/uL) | 0.9-9.3 | 3.75 | 1.92 | 3.47 | 2.68 | 1.096 | 0.0632 |
| MO (K/uL) | 0.0-0.4 | 0.14 | 0.12 | 0.11 | 0.17 | 0.073 | 0.0405 |
| NE (%) | 6.6-38.9 | 36.70 | 31.63 | 33.52 | 28.25 | 6.687 | 0.7516 |
| LY (%) | 55.8-91.6 | 60.36 | 62.64 | 63.30 | 65.56 | 6.787 | 0.7606 |
| MO (%) | 0.0-7.5 | 2.30 | 3.77 | 2.02 | 4.63 | 2.517 | 0.7941 |

| | | | |
|---|---|---|---|
| Hematological Abnormalities | Normal | Normal | Normal |

Key (Sample ID and results)
72—72 h post-treatment
n—NAM (250 mg/kg/d)
p—PBS treatment (control)
1, 2, 3, 4 . . . mouse number
HEMAVET 950FS, DREW Scientific Inc, Oxford, CT
MASCOT HEMATOLOGY PROFILE
Species: mouse (129/Sv-E, wildtype)
Date of test: Mar. 30, 2011

### Example 33

### Ex Vivo Hematological Study

Blood from human donors (no antibiotics within the prior 2 weeks, no immune-boosting supplements, and otherwise healthy) was collected in ETDA tubes. CBC was performed on each blood sample at time zero. Each blood sample was then treated ex vivo with NAM (1 mM) or PBS (control) for 24 h in 6-well non-treated plates at 37 C, 5% CO₂ and 95%% humidity with gentle rocking. After 24 h of treatment, CBCs were performed on each blood sample. No differences in CBCs were observed between NAM- and PBS-treated blood and untreated blood. (Table 3)

TABLE 3
CBC and DIFF, AUTOMATED: Beckman Coulter ® LH
1500 Series Hematology Automation

| | Result | | |
|---|---|---|---|
| | | t = | t = |
| | t = | 24 h | 24 h |
| Ref. Range | 0 h | (PBS) | (1 mM NAM) |

Human Donor#1^{a}

| Routine Blood | | | | | |
|---|---|---|---|---|---|
| Count | | | | | |
| WBC COUNT* | 4-11 | (1000/UL) | 9.2 | 8.9 | 8.9 |
| Automated | | | | | |
| Differential | | | | | |
| POLYS | % | | 62 | 67 | 61 |
| ABS POLYS | 1.8-8.0 | (1000/UL) | 5.7 | 6.0 | 5.4 |
| MONOS | % | | 6 | 5 | 7 |
| ABS MONOS | <0.8 | (1000/UL) | 0.6 | 0.4 | 0.6 |

Human Donor#2^{b}

| Routine Blood | | | | | |
|---|---|---|---|---|---|
| Count | | | | | |
| WBC COUNT* | 4-11 | (1000/UL) | 5.6 | 5.6 | 5.6 |
| Automated | | | | | |
| Differential | | | | | |
| POLYS | % | | 59 | 66 | 51 |
| ABS POLYS | 1.8-8.0 | (1000/UL) | 3.3 | 3.7 | 2.9 |
| MONOS | % | | 8 | 8 | 9 |
| ABS MONOS | <0.8 | (1000/UL) | 0.4 | 0.4 | 0.5 |

Human Donor#3^{c}

| Routine Blood | | | | | |
|---|---|---|---|---|---|
| Count | | | | | |
| WBC COUNT* | 4-11 | (1000/UL) | 7.3 | 6.8 | 6.9 |
| Automated | | | | | |
| Differential | | | | | |
| POLYS | % | | 70 | 72 | 76 |
| ABS POLYS | 1.8-8.0 | (1000/UL) | 5.1 | 4.9 | 5.2 |
| MONOS | % | | 5 | 5 | 4 |
| ABS MONOS | <0.8 | (1000/UL) | 0.4 | 0.3 | 0.2 |

Human Donor#4^{d}

| Routine Blood | | | | | |
|---|---|---|---|---|---|
| Count | | | | | |
| WBC COUNT* | 4-11 | (1000/UL) | 7.6 | 6.9 | 6.7 |
| Automated | | | | | |
| Differential | | | | | |
| POLYS | % | | 64 | 70 | 63 |
| ABS POLYS | 1.8-8.0 | (1000/UL) | 4.9 | 4.8 | 4.2 |
| MONOS | % | | 8 | 1 | 2 |
| ABS MONOS | <0.8 | (1000/UL) | 0.6 | 0.1 | 0.1 |

Human Donor#5^{e}

| Routine Blood | | | | | |
|---|---|---|---|---|---|
| Count | | | | | |
| WBC COUNT* | 4-11 | (1000/UL) | 6.9 | 5.9 | 6.1 |
| Automated | | | | | |
| Differential | | | | | |
| POLYS | % | | 67 | 60 | 70 |
| ABS POLYS | 1.8-8.0 | (1000/UL) | 4.6 | 3.5 | 4.3 |
| MONOS | % | | 8 | 3 | 2 |
| ABS MONOS | <0.8 | (1000/UL) | 0.5 | 0.2 | 0.1 |

*WBC COUNT: ABS POLYS + ABS LYMPHS + ABS MONOS + ABS
EOS + ABS BASOS
^{a}Female, 37 yo, Asian
^{b}Male, 36 yo, Black
^{c}Female, 69 yo, caucasian
^{d}Male, 48 yo, Caucasian
^{e}Female, 51 yo, Asian

| | P-value* | | |
|---|---|---|---|
| | WBC | ABS | ABS |
| | COUNT | POLYS | MONOS |
| t = 0 h vs. t = 24 h PBS | 0.0426 | 0.6261 | 0.0628 |
| t = 0 h vs. t = 24 h NAM | 0.0441 | 0.0669 | 0.1543 |
| t = 24 h PBH vs. t = 24 h NAM | 0.7781 | 0.5932 | 0.7489 |

*Two-tailed, Paired Students t Test

| | P-value* | |
|---|---|---|
| | POLYS % | MONOS % |
| t = 0 h vs. t = 24 h PBS | 0.3642 | 0.1443 |
| t = 0 h vs. t = 24 h NAM | 0.9364 | 0.2396 |
| t = 24 h PBS vs. t = 24 h NAM | 0.5589 | 0.5415 |

*Two-tailed, Paired Students t Test

### REFERENCES

1. Lowy, F. D. Staphylococcus aureus infections. N. Eng. J. Med. 339, 520-532 (1998).
2. Miller, L. G. & Kaplan, S. L. Staphylococcus aureus: a community pathogen. Infect. Dis. Clin. North. Am. 23, 35-52 (2009).
3. Delco, F. R., Otto, M., Kreiswirth, B. N. & Chambers, H. F. Community-associated meticillin resistant Staphylococcus aureus. Lancet. 375, 1557-1568 (2010).
4. Strauss, R. G., Bove, K. E., Jones, J. F., Mauer, A. M. & Fulginiti, V. A. An anomaly of neutrophil morphology with impaired function. N. Engl. J. Med. 290, 478-484 (1974).
5. Komiyama, A., Morosawa, H., Nakahata, T., Miyagawa, Y. & Akabane, T. Abnormal neutrophil maturation in a neutrophil defect with morphologic abnormality and impaired function. J. Pediatr. 94, 19-25 (1979).
6. Ambruso, D. R. et al. Defective bactericidal activity and absence of specific granules in neutrophils from a patient with recurrent bacterial infections. J. Clin. Immunol. 4, 23-30 (1984).
7. Shiohara, M. et at Phenotypic and functional alterations of peripheral blood monocytes in neutrophil-specific granule deficiency. J. Leukoc. Biol. 75, 190-197 (2004).
8. Lekstrom-Himes, J. A., Dorman, S. E., Kopar, P., Holland, S. M. & Gallin, J. I. Neutrophil-specific granule deficiency results from a novel mutation with loss of function of the transcription factor CCAAT/enhancer binding protein epsilon. J. Exp. Med. 189, 1847-1852 (1999).
9. Gombart, A. F. et al. Neutrophil-specific granule deficiency: homozygous recessive inheritance of a frameshift mutation in the gene encoding transcription factor CCAAT/enhancer binding protein-epsilon. Blood. 97, 2561-2567 (2001).
10. Chumakov, A. M. et al. Cloning of the novel human myeloid-cell-specific C/EBP-epsilon transcription factor. Mol. Cell. Biol. 17, 1375-1386 (1997).
11. Yamatake, R. et al. CCAAT/enhancer binding protein epsilon is preferentially up-regulated during granulocytic differentiation and its functional versatility is determined by alternative use of promoters and differential splicing. Proc. Natl. Acad. Sci. USA. 94, 6462-6467 (1997).
12. Yamanaka. R. et al. Impaired granulopoiesis, myelodysplasia, and early lethality in CCAAT/enhancer binding protein e-deficient mice. Proc. Natl. Acad. Sci. U.S.A. 94, 13187 (1997).
13. Williams, S. C. et al. C/EBPepsilon is a myeloid-specific activator of cytokine, chemokine, and macrophage-colony-stimulating factor receptor genes. J. Biol. Chem. 273, 13493-13501 (1998).
14. Lekstrom-Himes, J. & Xanthopoulos, K. G. CCAAT/enhancer binding protein epsilon is critical for effective neutrophil-mediated response to inflammatory challenge. Blood. 93, 3096-3105 (1999).
15. Verbeek, W. et al. Myeloid transcription factor C/EBPepsilon is involved in the positive regulation of lactoferrin gene expression in neutrophils. Blood. 94, 3141-3150 (1999).
16. Tavor, S. et al. Macrophage functional maturation and cytokine production are impaired in C/EBPepsilon-deficient mice. Blood. 99, 1794-1801 (2002).
17. Gombart, A. F. et al. Aberrant expression of neutrophil and macrophage-related genes in a murine model for human neutrophil-specific granule deficiency. J. Leukoc. Biol. 78, 1153-1165 (2005).
18. Ceseña, T. I., Cardinaux, J. R., Kwok, R. & Schwartz, J. CCAAT/enhancer-binding protein (C/EBP) beta is acetylated at multiple lysines: acetylation of C/EBPbeta at lysine 39 modulates its ability to activate transcription. J. Biol. Chem. 282, 956-967 (2007).
19. Norton, V., Marvin, K., Yau, P. & Bradbury, E. Nucleosome linking number change controlled by acetylation of histones 113 and H4. J. Biol. Chem. 265, 19848-19852 (1990).
20. Lee, D. Y, Hayes, J. J., Pruss, D. & Wolffe, A. P. A positive role for histone acetylation in transcription factor access to nucleosomal DNA. Cell 72, 73-84 (1993).
21. Rundlett, S. E. et al. HDA1 and RPD3 are members of distinct yeast histone deacetylase complexes that regulate silencing and transcription. Proc. Natl Acad. Sci. USA 93, 14503-14508 (1996).
22. Kouzarides, T. Acetylation: a regulatory modification to rival phosphoryation? EMBO. J. 19, 1176-1179 (2000).
23. Glozak, M. A., Sengupta, N., Zhang, X. & Seto, E. Acetylation and deacetylation of non-histone proteins. Gene. 363, 15-23 (2005).
24. Bitterman, K. J., Anderson, R. M., Cohen, H. Y., Latorre-Esteves, M. & Sinclair, D. A. Inhibition of silencing and accelerated aging by nicotinamide, a putative negative regulator of yeast sir2 and human SIRT1. J. Biol. Chem. 277, 45099-45107 (2002).
25. Sauve, A. A. & Schramm, V. L. Sir2 regulation by nicotinamide results from switching between base exchange and deacetylation chemistry. Biochemistry. 42, 9249-9256 (2003).
26. Jackson, M. D., Schmidt, M. T., Oppenheimer, N. J. & Denu, J. M. Mechanism of nicotinamide inhibition and transglycosidation by Sir2 histone/protein deacetylases. Biol. Chem. 278, 50985-50998 (2003).
27. Porcu, M. & Chiarugi, A. The emerging therapeutic potential of sirtuin-interacting drugs: from cell death to lifespan extension. Trends Pharmacol. Sci. 26, 94-103 (2005).
28. Koren, H. S., Anderson, S. J, Larrick, J. W. In vitro activation of a human macrophage-like cell line. Nature. 1979 279, 328-331 (1979).
29. Passmore, J. S., Lukey, P. T. & Ress, S. R. The human macrophage cell line U937 as an in vitro model for selective evaluation of mycobacterial antigen-specific cytotoxic T-cell function. Immunology. 102, 146-156 (2001).
30. Bernier, J. et al. Pharmacokinetics of nicotinamide in cancer patients treated with accelerated radiotherapy: the experience of the Co-operative Group of Radiotherapy of the European Organization for Research and Treatment of Cancer. Radiother. Oncol. 48, 123-133 (1998).
31. Todd, P. A. & Goa, K. L. Interferon gamma-1b. A review of its pharmacology and therapeutic potential in chronic granulomatous disease. Drugs 43, 111-122 (1992).
32. Marciano, B. E. et al. Long-term interferon-gamma therapy for patients with chronic granulomatous disease. Clin. Infect. Dis. 39, 692-699 (2004).
33. Vilcek, J. Fifty years of interferon research: aiming at a moving target. Immunity. 25, 343-348 (2006).
34. Gombart, A. F. et al. Low plasma level of cathelicidin antimicrobial peptide (hCAP18) predicts increased infectious disease mortality in patients undergoing hemodialysis. Clin. Infect. Dis. 48, 418-424 (2009).
35. Klaidman, L. K., Mukherjee, S. K., Hutchin, T. P. & Adams, J. D. Nicotinamide as a precursor for NAD+ prevents apoptosis in the mouse brain induced by tertiary-butylhydroperoxide. Neurosci. Lett. 206, 5-8 (1996).
36. Skokowa, J. et al. NAMPT is essential for the G-CSF-induced myeloid differentiation via a NAD(+)-sirtuin-1-dependent pathway. Mat. Med. 15, 151-158 (2009).
37. Lomax, K. J. Selective defect in myeloid cell lactoferrin gene expression in neutrophil-specific granule deficiency. J. Clin. Invest. 83, 514-519 (1989).
38. Horsman, M. R., Hoyer, M., Honess, D. J., Dennis, I. F. & Overgaard, J. Nicotinamide pharmacokinetics in humans and mice: a comparative assessment and the implications for radiotherapy. Radiother. Oncol. 27, 131-139 (1993).
39. Kaneko, S. et al. Protecting axonal degeneration by increasing nicotinamide adenine dinucleotide levels in experimental autoimmune encephalomyelitis models. J. Neurosci. 26, 9794-9804 (2006).
40. Murray, M. F. Nicotinamide: an oral antimicrobial agent with activity against both Mycobacterium tuberculosis and human immunodeficiency virus. Clin. Infect. Dis. 36, 453-460 (2003).
41. Liu, G. Y. et al. Staphylococcus aureus golden pigment impairs neutrophil killing and promotes virulence through its antioxidant activity. J. Exp. Med. 202, 209-215 (2005).
42. Bunce, C., Wheeler, L., Reed, G., Musser, J., Barg, N, Murine model of cutaneous infection with gram-positive cocci. Inject. Immun. 60, 2636-2640 (1992).
43. Tseng, C. W. et al. Staphylococcus aureus Panton-Valentine leukocidin contributes to inflammation and muscle tissue injury. PLoS One. 4, e6387 (2009).
44. Savov, J. D., Gavett S. H., Brass, D. M., Costa, D. L. & Schwartz, D. A. Neutrophils play a critical role in development of LPS-induced airway disease. Am. J. Physiol. Lung. Cell. Mol. Physiol. 283, 952-962 (2002).
45. Gao, X. P. et al. Inactivation of CD11b in a mouse transgenic model protects against sepsis-induced lung PMN infiltration and vascular injury. Physiol. Genomics. 21, 230-242 (2005).
46. Park, D. J. et al. CCAAT/enhancer binding protein epsilon is a potential retinoid target gene in acute promyelocytic leukemia treatment. J. Clin. Invest. 103, 1399-1408 (1999).
47. Liu, C. I., et al. A cholesterol biosynthesis inhibitor blocks Staphylococcus aureus virulence. Science. 319, 1391-1394 (2008).
48. Maiese, K., Chong, Z. Z., Hou, J. & Shang, Y. C. The vitamin nicotinamide: translating nutrition into clinical care. Molecules. 14, 3446-3485 (2009).
49. Kaanders, J. H. et al. Administration of nicotinamide during a five- to seven-week course of radiotherapy: pharmacokinetics, tolerance, and compliance. Radiother. Oncol. 43, 67-73 (1997).
50. Stratford, M. R. et al. Pharmacokinetics of nicotinamide and its effect on blood pressure, pulse and body temperature in normal human volunteers. Radiother. Oncol. 25, 37-42 (1992).
51. Wurtele, H. et al. Modulation of histone H3 lysine 56 acetylation as an antifungal therapeutic strategy. Nat. Med. 16, 774-780 (2010).

What is claimed is:

1. A method, comprising:
providing a composition that upregulates the expression of CCAAT/enhancer binding protein epsilon (C/EBPε), and
administering a therapeutic dose of the composition to an individual having an infection caused by a pathogen, whereby an enhanced immune response to the infection results in the individual.

2. The method of claim 1, wherein the composition comprises vitamin B3 or an analog, derivative or salt thereof.

3. The method of claim 1, wherein the pathogen is selected from the group consisting of: parasites, fungi, bacteria, viruses, or combinations thereof.

4. The method of claim 1, wherein the pathogen is selected from the group consisting of: Staphylococcus aureus (S. aureus), methicillin-resistant S. aureus, Vancomycin resistant Enterococcus, C. difficile, B. cepacia, influenza, rhinovirus, Epstein barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof.

5. The method of claim 1, wherein the individual is a mammal.

6. The method of claim 1, wherein the individual is a human.

7. A method, comprising:
providing a composition that upregulates the expression of CCAAT/enhancer binding protein epsilon (C/EBPε), and
administering a prophylactic dose of the composition to an individual, whereby the likelihood of developing a severe pathogenic infection in the individual is reduced.

8. The method of claim 7, wherein the composition comprises vitamin B3 or an analog, derivative or salt thereof.

9. The method of claim 7, wherein the pathogenic infection is caused by a pathogen selected from the group consisting of: parasites, fungi, bacteria, viruses, or combinations thereof.

10. The method of claim 7, wherein the pathogenic infection is caused by a pathogen selected from the group consisting of: Staphylococcus aureus (S. aureus), methicillin-resistant S. aureus, Vancomycin resistant Enterococcus, C. difficile, B. cepacia, influenza, rhinovirus, Epstein barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof.

11. The method of claim 7, wherein the individual is a mammal.

12. The method of claim 7, wherein the individual is a human.

13. The method of claim 7, wherein the composition is administered as part of a parenteral nutrition regimen.

14. The method of claim 7, wherein the individual is a neonate or other patient that cannot eat on his or her own.

15. A method, comprising:
providing interferon-gamma, and
administering a therapeutic dose of interferon-gamma to an individual having a pathogenic infection and a defective innate immune response thereto, whereby the severity of the pathogenic infection is reduced.

16. The method of claim 15, wherein the pathogen is selected from the group consisting of: parasites, fungi, bacteria, viruses, or combinations thereof.

17. The method of claim 15, wherein the pathogen is selected from the group consisting of: Staphylococcus aureus (S. aureus), methicillin-resistant S. aureus, Vancomycin resistant Enterococcus, C. difficile, B. cepacia, influenza, rhinovirus, Epstein barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof.

18. The method of claim 15, wherein the individual is a mammal.

19. The method of claim 15, wherein the individual is a human.

20. The method of claim 15, wherein the individual has neutrophil-specific granule deficiency.

21. A method, comprising:
providing interferon-gamma, and
administering a prophylactic dose of interferon-gamma to an individual with a defective innate immune response to a pathogen, whereby the likelihood of developing a severe pathogenic infection is reduced.

22. The method of claim 21, wherein the pathogen is selected from the group consisting of: parasites, fungi, bacteria, viruses, or combinations thereof.

23. The method of claim 21, wherein the pathogen is selected from the group consisting of: Staphylococcus aureus (S. aureus), methicillin-resistant S. aureus, Vancomycin resistant Enterococcus. C. difficile, B. cepacia, influenza, rhinovirus, Epstein barr virus, cytomegalovirus, adenovirus, parainfluenza virus, rotavirus, candida, ESBL gram negative pathogens, S. epidermidis, Pseudomonas, Enterobacter, vancomycin resistant Enterobacter, E. coli, Salmonella, Streptococcus, Chlamydia, Campylobacter, Helicobacter, Mycobacteria; antibiotic resistant gram negative pathogens such as acinetobacter; pathogens from Example 31 (Table 1), or combinations thereof.

24. The method of claim 21, wherein the individual is a mammal.

25. The method of claim 21, wherein the individual is a human.

26. The method of claim 21, wherein the individual has neutrophil-specific granule deficiency.

27. A method, comprising:
providing a composition that upregulates the expression of CCAAT/enhancer binding protein epsilon (C/EBPε), and
administering a therapeutic dose of the composition to an individual having an inflammatory condition, whereby an increased anti-inflammatory response results in the individual.

28. The method of claim 27, wherein the composition is Vitamin B3 or an analog, derivative or salt thereof.

29. The method of claim 27, wherein the upregulation of C/EBPEε increases interleukin 10 (IL-10) function.

30. The method of claim 29, wherein the increased IL-10 function results in anti-inflammatory mediation of an inflammatory and/or autoimmune condition selected from the group consisting of: atherosclerosis, inflammatory bowel diseases, multiple sclerosis, rheumatoid arthritis, asthma, bacterial sepsis, Kawasaki's disease, atopic dermatitis, and other rheumatologic conditions.

31. A kit comprising:
a volume of a composition that upregulates the expression of CCAAT/enhancer binding protein epsilon (C/EBPε), and
instructions for the use of said composition in the treatment of a disease condition in a mammal.
